(19) United States
(12) Patent Application Publication
(10) Pub. No.: US 2025/0381157 A1
(43) Pub. Date: Dec. 18, 2025

(54) ANTIMICROBIAL COMPOUNDS

(71) Applicant: AKTHELIA PHARMACEUTICALS, REYKJAVIK (IS)

(72) Inventors: Egill MÁSSON, Reykjavik (IS); Guðmundur Hrafn GUÐMUNDSSON, REYKJAVIK (IS); Iwona Teresa MYSZOR, REYKJAVIK (IS); Kurt Roger STRÖMBERG, Solna (SE)

(21) Appl. No.: 18/874,838

(22) PCT Filed: Jun. 9, 2023
(86) PCT No.: PCT/EP2023/065555

§ 371 Date: Dec. 13, 2024

(30) Foreign Application Priority Data
Jun. 13, 2022 (GB) 2208649.0

(51) Int. Cl. A61K 31/167; A61K 31/198; A61K 31/4406; A61K 31/59; A61K 45/06; A61P 31/04; C07C 233/65

(52) U.S. Cl. CPC A61K 31/167; A61K 31/198; A61K 31/4406; A61K 31/59; A61K 45/06; A61P 31/04; C07C 233/65

(57) ABSTRACT

The invention relates to phenylene diamine derivatives with certain pharmacological properties resulting from enhancement of epithelial barrier function and/or blocking bacterial translocation through the epithelial barrier. The compounds find use in the treatment of various conditions, including conditions involving translocation of pathogens from the gastrointestinal tract into underlying tissues and vasculature, for example febrile neutropenia, intestinal tissue inflammation, bacteremia and sepsis.

### TECHNICAL FIELD

The invention relates to phenylene diamine derivatives with certain pharmacological properties resulting from enhancement of epithelial barrier function and/or blocking bacterial translocation through the epithelial barrier. The compounds find use in the treatment of various conditions, including conditions involving translocation of pathogens from the gastrointestinal tract into underlying tissues and vasculature, for example febrile neutropenia, intestinal tissue inflammation, bacteremia and sepsis.

### BACKGROUND ART

Damage to the epithelial barrier (mucosal barrier), such as in the gastrointestinal tract, can permit the translocation of pathogens through the barrier, leading to diseases such as neutropenia, febrile neutropenia, intestinal tissue inflammation, bacteremia and sepsis.

The epithelial barrier can be damaged in multiple ways, such as by chemical means following cancer treatment/chemotherapy, organ transplant, and infection by intestinal pathogens, leading to dysbiosis of native microbiota (which can be brought on by direct-acting antimicrobials), or mucositis.

Damage to the epithelial barrier can be particularly common in subjects with low neutrophil count (neutropenia), such as in patients undergoing cytotoxic cancer treatment, or treated with immunosuppressants. Low neutrophil count resulting from cytotoxic chemotherapy is associated with an increased risk of severe bacterial and fungal infections. This has now been causally linked to mucositis-cytotoxic damage to the lining of the GI tract and other mucosa, which leads the immunocompromised host to be vulnerable to invasion by infectious pathogens from their own microbiota.

Damage to the epithelial barrier in patients with neutropenia can lead to febrile neutropenia. Febrile neutropenia is the most common (˜1% of chemo- and radiotherapy patients) and serious, life-threatening complication associated with patients receiving chemotherapy for cancer or who are immunocompromised to avoid transplant rejection. Febrile neutropenia is often a limiting factor in treatment dosage and it has significant mortality rates—5% up to 50% in high-risk populations, with >60,000 hospitalizations and >4,000 deaths annually costing ˜$2.8B (2012) in the US alone, >8% of all cancer-related hospitalization costs (see Original Articles Epidemiology|Volume 23, Issue 7, P1889-1893, Jul. 1, 2012).

The current treatment paradigm of prophylaxis with fluoroquinolone broad-spectrum antibiotics is unpopular with oncologists, not only because of drug toxicity, but also because it leads to increased occurrence of drug-resistant bacteria and dysbiosis of the intestinal microbiota, which further encourages colonisation by opportunistic pathogens. An antimicrobial approach is also fundamentally ineffective because it does not halt translocation of infectious organisms through the GI tract wall into underlying tissues and vasculature.

WO2015063694 (Akthelia) relates to benzoylated phenylenediamines and their use in treating microbial infections.

Despite the above disclosures, it will be appreciated that the provision of compounds or combinations of compounds for use in treating diseases caused by or resulting from damage to the epithelial barrier integrity and/or bacterial translocation through the epithelial barrier would provide a contribution to the art.

### SUMMARY OF INVENTION

The inventors applied a therapeutic strategy based on depth of field-leading cell biology expertise, to eliminate bacterial translocation and enhance the innate immunologic barrier resistance of the GI tract.

This was achieved in part through providing compounds that are capable of upregulating endogenous proteins that are naturally expressed within epithelia and improving tight junction signalling in epithelial barrier, which can act to contain the microbiota within the gut lumen in a healthy individual.

Data disclosed herein shows that compounds of the invention unexpectedly and effectively knock down pathogen translocation in relevant murine models into multiple organs and systems, suggesting translatable survival benefit. The inventors believe this may be achieved by compounds of the invention exerting an effect on the tight junctions, such as through intersecting signal pathways, leading to very efficient blocking of translocation, as well as activating the host-defence system.

To the inventors' knowledge, it was not previously known whether compounds capable of upregulating endogenous proteins would also separately be capable of blocking pathogen translocation. In fact, certain inducers of endogenous, antimicrobial peptides have previously been shown to downregulate proteins essential in the tight junction of the epithelial barrier. See e.g. Yolanda M. Jacobo-Delgado et al; Peptides, Volume 142, 2021, 170580, https://doi.org/10.1016/i.peptides.2021.170580; and Hatakeyama S et al, J Periodontal Res. 2010 April; 45(2):207-15. doi: 10.1111/j.1600-0765.2009.01219.x.) The claimed invention allows for a new standard of care treatment with potential to replace prophylactic use of antibiotics and antifungals in patients at risk for febrile neutropenia, with reduced risk of SAEs, AMR, and microbiome dysbiosis.

This highly differentiated approach has broad application and can help the treatment of diseases described herein, as well as address the growing threat of antibiotic bacterial resistance worldwide.

Certain advantages that may be displayed by the claimed invention are as follows: (1) combined strengthening of epithelial barrier (tight junctions) and upregulation of host defence peptides in epithelial cells and macrophages; (2) broad spectrum therapy with a different mode of action from antibiotics—immunomodulation that induces production of host defence peptides which attenuate bacteria; (3) pathogens are unlikely to become resistant, as production of multiple host defence microbial factors is induced. This is in contrast with antibiotics, which act directly on the microbes, thus quickly selecting for resistant strains; (4) minimal impact on natural microbiota.

Accordingly, the invention provides compounds which are effective in improving or restoring epithelial barrier function and/or preventing or reducing microbial translocation through the epithelial barrier of an animal. In preferred embodiments, the epithelial barrier is the intestinal or gastrointestinal barrier. The compounds are benzoylated phenylenediamines or derivatives or analogs thereof, as described in more detail hereinafter.

Preferred compounds are N-(2-aminophenyl)-4-{2-[(prop-2-yn-1-yl)oxy]acetamido}benzamide (“Compound 1”), N-(2-aminophenyl)-4-[(methyl{[(prop-2-yn-1-yl)oxy]acetyl}amino)methyl]benzamide (“Compound 2.1”), N-(2-aminophenyl)-4-{[N-methyl-2-(2-propanamidoethoxy)acetamido]methyl}benzamide (“Compound 2.2”), 4-[(2-aminophenyl)carbamoyl]phenyl hex-5-ynoate (“Compound 2.3”), and Pyridin-3-ylmethyl (4-((2-aminophenyl)-carbamoyl)benzyl)carbamate (“Entinostat”).

The present invention provides for the use of the compounds described herein for the treatment of diseases disclosed herein. Preferred microbial targets and diseases targeted by the present invention are described hereinafter.

This effectiveness of this class of compounds in preventing microbial translocation (such as bacterial translocation and/or fungal translocation) through epithelial barriers is unexpected and implies that these compounds and their analogs may work via different or additional stimulatory mechanisms to some previous compounds that e.g. exclusively stimulate the innate antimicrobial defence peptide system.

Aspects of the invention are methods for treatment (including prophylaxis) of diseases described herein in an animal using the compounds described herein.

The present invention further provides a compound as defined herein for use as a medicament for treating the diseases described herein in humans and other animals by improving or restoring epithelial barrier function.

In yet a further aspect, the invention provides a pharmaceutical composition for use in the methods described herein, comprising an active ingredient being at least one compound of the invention, and typically at least one pharmaceutically acceptable excipient.

In yet a further aspect, the invention provides use of compounds of the invention in the preparation of a medicament for use in the methods described herein.

In one aspect, the invention provides compounds of general formula (I), for use in a method of treatment or prophylaxis of a disease or condition in an animal that would benefit from enhancement or restoration of epithelial barrier function, wherein administration of the compound improves, restores or maintains epithelial barrier function in the animal, wherein the compound is defined by the following formula:

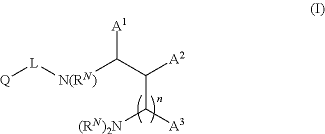

wherein:
Q is selected from Q1, Q2, Q3, Q4, Q5 and Q6:

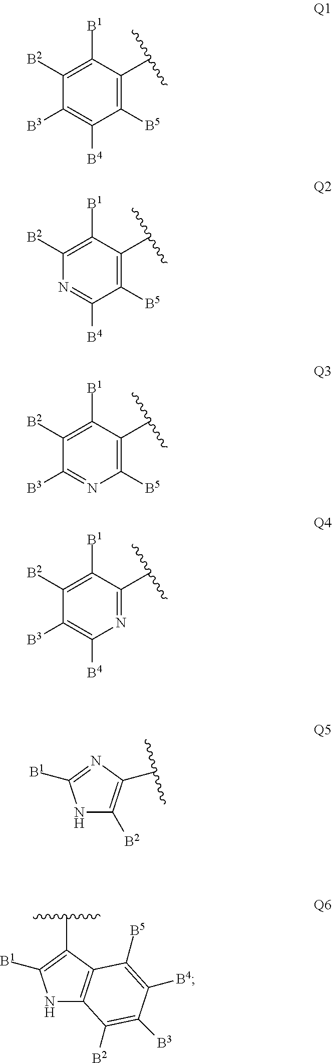

n is 0 or 1;
L is selected from —(CH₂)ₘ—, —C(═O)—, —(CH₂)ₘ—C(═O)—, —O—(CH₂)ₘ—C(═O)—, —O—C(═O)—(CH₂)ₘ—(C=O)—, —NH—C(═O)—, —NR—C(═O)—, —NH—(CH₂)ₘ—C(═O)—, —NR—(CH₂)ₘ—C(═O)—, —NH—C(═O)—(CH₂)ₘ—C(═O)—, —NR—C(═O)—(CH₂)ₘ—C(═O)—, —C(═O)—NH—(CH₂)ₘ—C(═O)—, and —(CH₂)ₘ—(CHR^{L})—C(═O)—, where m is an integer from 1 to 4;
A¹ and A², together with the atoms to which they are bound, form an optionally substituted C₆₋₁₄aryl or heteroaryl group;
A³, if present, is selected from H and optionally substituted C₁₋₄alkyl;
R^{N} is selected from H and optionally substituted³ C₁₋₄alkyl;
one of B¹, B², B³, B⁴, and B⁵ is a group of formula —X—R^{X} and the others are independently selected from H and R^{B};
   wherein each —R^{B} is independently selected from halogen, —CF₃, —R, —OH, —OR, —OCF₃, —C(═O)OH, —C(═O)OR, —C(═O)R, —OC(═O)R, —NH₂, —NHR, —NR₂, —NO₂, —C(═O)NH₂, —C(═O)NHR, C(═O)NR₂, —S(═O)R, —S(═O)₂R, —S(═O)₂NR₂, or —CN;
X is selected from a covalent bond or C₁₋₃alkylene;
R^{X} is selected from —H, R^{XX} or R^{XY};
   wherein:
   R^{XX} is halogen, —CF₃, —OH, —OR, —OCF₃, —C(═O)OH, —NO₂, —NH₂, —NHR, —NR₂, —C(═O)NH₂, —C(═O)NR₂, —S(═O)R, —S(═O)₂R, —S(═O)₂NR₂, or —CN; and
   R^{XY} is a group of formula -L^{X}-R^{YY};
   wherein L^{X} is selected from:
   —NH—C(═O)—O—, —NH—C(═O)—NH—, —NH—C(═O)—, —NR—C(═O)—, —O—C(═O)—NH—, —O—C(═O)—O—, —O—(C=O)—, —C(═O)—NH—, —C(═O)—O—, —C(═O)—;
   and R^{YY} is selected from C₁₋₄alkyl, C₃₋₆cycloalkyl, —C₆₋₁₄aryl, -L^{Y}-C₆₋₁₄aryl, -L^{Y}-O—C₆₋₁₄aryl, —C₅₋₆heteroaryl, -L^{Y}-C₅₋₆heteroaryl, -L^{Y}-O—C₅₋₆heteroaryl, -L^{Y}-O-L^{Y}-C=N, -L^{Y}-O-L^{Y}-C=R, -L^{Y}-O-L^{Y}-C≡CH, -L^{Y}-O-L^{Y}-NR^{N}_C(═O)—R, -L^{Y}-O-L^{Y}-NH—C(═O)—R, -L^{Y}-O-L^{Y}-NR—C(═O)—R, -L^{Y}-C≡N, -L^{Y}-C=R, -L^{Y}-C≡CH,
      wherein -L^{Y}- is C₁₋₃alkylene and wherein each of said groups is optionally substituted;
R^{L} is selected from halogen, —R^{LL}, —CF₃, —OH, —OR^{LL}, —NO₂, —NH₂, —NHR^{LL}, —NR₂, —NH—C(═O)—R^{LL}, —NH—C(═O)—O—R^{LL} wherein R^{LL} is selected from —C₁₋₄alkyl, —C₃₋₆cycloalkyl, -Ph, -L^{L}-Ph, —C₅₋₆heteroaryl, -L^{L}-C₅₋₆heteroaryl wherein -L^{L}- is C₁₋₃alkylene.
and wherein each R is independently C₁₋₄alkyl.

In a further aspect and embodiment, the invention provides compounds of general formula (I), for use in a method of treatment or prophylaxis of a disease or condition in an animal that would benefit from preventing or reducing microbial translocation through the epithelial barrier of the animal, wherein administration of the compound prevents or reduces microbial translocation through the epithelial barrier of the animal.

The diseases or conditions that would benefit from preventing or reducing microbial translocation through the epithelial barrier of the animal, or that would benefit from enhancement or restoration of epithelial barrier function, include the particular diseases disclosed herein.

In some particularly preferred embodiments, the compound is according to formula (Ia):

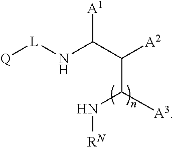

Some aspects and embodiments of the invention will now be described in more detail.

### Further Description and Preferences

### Methods of Treatment

Compounds described herein may be novel per se. Thus, aspects of the invention extend to those compounds per se, in addition to their uses in the therapeutic methods described herein. The compounds may be used alone or as adjunctive therapy.

The compounds of the invention may treat and/or prevent the diseases described herein in any combination thereof, or alone.

The term “treatment,” as used herein in the context of treating a disorder, pertains generally to treatment and therapy, whether of a human subject or another animal (e.g. mammal), in which some desired therapeutic effect is achieved, for example, the inhibition of the progress of the disorder, and includes a reduction in the rate of progress, a halt in the rate of progress, alleviation of symptoms of the disorder, amelioration of the disorder, and cure of the disorder.

The term “treatment” includes combination treatments and therapies, in which two or more treatments or therapies are combined, for example, sequentially or simultaneously.

The agents (i.e. the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g. 1, 2, 3, 4 or more hours apart, or even longer periods apart where required), the precise dosage regimen being commensurate with the properties of the therapeutic agent(s) as described herein, including their synergistic effect.

Treatment as a prophylactic measure (i.e., prophylaxis) is also included. For example, use with patients who have not yet developed the disorder, but who are at risk of developing the disorder, is encompassed by the term “treatment.”

“Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

The methods and compositions of the present invention will be understood to also have utility in aquaculture, veterinary and animal husbandry applications for companion animals, farm animals, and ranch animals. These applications include but are not limited to treating, preventing or counteracting microbial diseases and conditions in fish, dogs, cats, cows, horses, deer and poultry including hen, turkey ducks, geese; as well as in household pets such as birds and rodents. For large animals, a suitable dose can be larger than the human approved amounts.

The compounds described herein can act by improving or restoring epithelial barrier function, i.e. strengthening the epithelial barrier, and thus can treat diseases and conditions that would benefit therefrom. To the inventors' knowledge, this is achieved by one or more of: promoting maintenance of tight junctions between epithelial cells; counteracting inflammation; and inducing the expression of antimicrobial peptides/proteins in epithelial cells.

Epithelial cells make up an important barrier separating the outside environment from the internal tissue milieu and have specific adaptations linked to their function. A single layer of polarized epithelial cells in the intestines separates internal tissue from the lumen of the gastrointestinal tract which contains high numbers of microbes especially in the colon. Prominent functions of intestinal epithelia, besides uptake of nutrients, involves them serving as an active barrier preventing bacteria migration to underlying tissues and secreting antimicrobial compounds.

Activities of epithelial cells are essential for maintaining the separation between host tissues and microbes and preserving host-microbe homeostasis. The epithelial cells are covered by a mucus protein layer containing antimicrobial effectors. The paracellular space between adjacent epithelial cells is sealed with interconnected junctional complexes with the tight junctions, composed of claudins/occludin. Decreased epithelial barrier function may thus manifest in some embodiments as decreased tight junction function, decreased junctional complex function, decreased claudin function, decreased occludin function, decreased claudin number, or decreased occluding number.

Improvement or restoration of epithelial barrier function and/or reduction of prevention of microbial translocation can be determined, for example, by measuring Colony Forming Unit (CFU) counts in the basal tissue, circulation and/or internal organs in in vivo animal models. Examples of internal organs relevant to the invention include highly vascularized organs including kidney, liver, and the spleen.

Improvement or restoration of epithelial barrier function can also be determined by effect on tight junction function, which itself can be determined by measuring trans-epithelial electrical resistance (TEER), or by analysing levels of tight junction proteins, such as occluding and claudin-1, by Western blot analysis. The skilled person would be aware of further methods in the art for measuring these properties.

In particularly preferred embodiments, the epithelial barrier is the intestinal epithelial barrier or gastrointestinal epithelial barrier.

The gastrointestinal tract (GI tract) of mammals is covered by a continuous sheet of epithelial cells (enterocytes) that is folded into villus projections and crypts. Within the base of the crypts, where the stem cells of the GI tract can be found, there are specialized, granular cells called Paneth cells. Both enterocytes and Paneth cells produce antimicrobial peptides. The enterocytes synthesize and secrete antimicrobial peptides into the gut lumen both constitutively and upon induction. The Paneth cells at the base of the intestinal crypts, secrete alpha-defensins into the cryptal well, resulting in concentrations estimated at mg/mL levels, which eventually flush into the gut lumen. Additionally stationary macrophages are also known to secrete antimicrobial peptides into the gut lumen and additional tissue sites when activated.

Other epithelial surfaces of the mammalian body also have such host defence secretion systems, including but not limited to the cornea, the lung, the kidney and the skin (see also WO2012/140504).

The stimulation of macrophages and epithelial cells and Paneth cells of the gastrointestinal tract and other epithelial surfaces of man and in other animals to secrete large quantities of naturally occurring broad-spectrum antimicrobial agents, including antimicrobial peptides such as defensins, HMP 1-4, LL-37, HBD1-4, and antimicrobial proteins such as lysozyme, transferrin, lactoferrin, phospholipases, and SLPI (secretory leukocyte protease inhibitor). The substances stored by the Paneth cells exhibit activity against a wide range of infectious agents including bacteria, protozoa, viruses, and fungi.

In doing so, the compounds of the invention act to contain the microbiota within the gut lumen like in a healthy individual. The natural microbiome has adapted to the milieu containing antimicrobial peptides, especially ones that bind bacteria and disrupt their membrane functions. These commensal bacteria make modified lipopolysaccharides (endotoxin) that do not bind as avidly to the positively charged antimicrobial peptides and thus requiring higher concentrations of the peptides for their bacteriostatic effects. In situation such as neutropenia, the concentration of antibacterial peptides is lower and less effective. Also as can be seen from the in vivo infection studies (FIG. 1), compound 1 is not eliminating the pathogen but is totally inhibiting its translocation, strongly suggesting that epithelial integrity is being maintained.

The epithelial cells targeted by the present invention may be any of these e.g. in the oral cavity, lung, trachea, urinary tract or kidney, stomach, upper GI tract (e.g. ileum) and lower GI tract (e.g. jejunum), and colon. Preferably however the invention is utilised for the treatment of diseases involving barrier function of the GI tract.

As mentioned, an important aspect of the invention provides methods for treating, preventing or counteracting microbial infections or any of the described diseases e.g. by administering a medicament comprising an effective amount of at least one compound of the invention, thereby improving or restoring epithelial barrier function and/or preventing or reducing microbial translocation. Observable procession of human infection, besides fever and swelling, can be monitored by doing cultures of blood samples, blood count and/or testing for bacterial products such as endotoxin. Additionally, measurable plasma proteins that are upregulated in infection include C-reactive protein, procalcitonin and inflammatory cytokines. Integrity of the intestinal epithelial layer can be assessed by the Intestinal Permeability (IP) test, also referred to as a “leaky gut” test, that measures ingested mannitol and lactulose levels in urine samples.

In some embodiments, the compounds are therefore capable of preventing microbial translocation from the gut of an animal to further locations in the body, such as basal tissue, the circulatory system, or other internal organs such as the kidney, liver or spleen, and vascular beds via the circulatory system.

Observable procession of human infection, besides fever and swelling, can be monitored by doing cultures of blood samples, blood count and/or testing for bacterial products such as endotoxin. Additionally, measurable plasma proteins that are upregulated in infection include C-reactive protein, procalcitonin and inflammatory cytokines. Integrity of the intestinal epithelial layer can be assessed by the Intestinal Permeability (IP) test, also referred to as a “leaky gut” test, that measures ingested mannitol and lactulose levels in urine samples.

The compounds described herein may be used in the treatment of febrile neutropenia. Febrile neutropenia may be defined by a single oral temperature measurement of >38.3° C. (>101° F.) or a temperature of ≥38.0° C. (>100.4° F.) sustained over 1 hour, with an absolute neutrophil count (ANC) of <500 cells/microlitre, or an ANC that is expected to decrease to <500 cells/microlitre over the next 48 hours. See e.g. Punnapuzha S, Edemobi P K, Elmoheen A. Febrile Neutropenia. [Updated 2022 Feb. 10]. In: StatPearls [Internet]. Treasure Island (FL): StatPearls Publishing; 2022 January. Available from: https://www.ncbi.nlm.nih.gov/books/NBK541102/.

Accordingly, in preferred embodiments the compounds described herein find use in treating patients with a low neutrophil count, such as with an ANC of <500 cells/microlitre, <450 cells/microlitre, <400 cells/microlitre, <350 cells/microlitre, <300 cells/microlitre, <250 cells/microlitre, <200 cells/microlitre, <150 cells/microlitre, <100 cells/microlitre, or <50 cells/microlitre.

In some embodiments, the febrile neutropenia is chemotherapy-induced febrile neutropenia. The risk of developing febrile neutropenia depends on the degree and duration of chemotherapy-induced neutropenia and on a number of patient factors, including age, comorbidity and serum albumin levels (Bodey et al, 1966; Meza et al, 2002; Lyman et al, 2005; Aapro et al, 2006). Thus, febrile neutropenia may be induced by any chemotherapy drugs known in the art. The patients to be treated by the invention may still be taking chemotherapy drugs. The patients to be treated by the invention may also have been treated with chemotherapy drugs in the past, such as 1 week, 1 month, 2 months, 3 months, 4 months, 5 months, 6 months, 1 year, 2 years, 3 years, 4 years or 5 years ago, or any amount of time in between.

In some embodiments, the compounds described herein therefore find use in treating patients who have been or are being treated for cancer. Their cancer treatment may be ongoing, or may have been administered in the past, such as 1 week, 1 month, 2 months, 3 months, 4 months, 5 months, 6 months, 1 year, 2 years, 3 years, 4 years or 5 years ago, or any amount of time in between.

Febrile neutropenia may also be a result of organ transplantation, wherein patients are administered with immunosuppressant drugs known in the art, that cause lower leukocyte numbers or depress their function. Many immunosuppressive drugs also affect epithelial cells due to the integrated function of the immune system with epithelial cell layers exposed to microorganisms (https://doi.org/10.1038/s12276-018-0126-x). Immunosuppressants also alter the gut microbiome that can lead to increased risk of infections (doi: 10.1097/QCO.0b013e3283630dd3).

In some embodiments, the compounds described herein therefore find use in treating patients on immunosuppressants who are the recipient of an organ transplant. In some preferred cases, the transplant may be a marrow transplant or a kidney transplant. The organ transplant may have been performed in the past, such as 1 week, 1 month, 2 months, 3 months, 4 months, 5 months, 6 months, 1 year, 2 years, 3 years, 4 years or 5 years ago, or any amount of time in between. The patients may be immunocompromised. The patients to be treated by the invention may still be taking immunosuppressant drugs. The patients to be treated by the invention may also have been treated with immunosuppressant drugs in the past, such as 1 week, 1 month, 2 months, 3 months, 4 months, 5 months, 6 months, 1 year, 2 years, 3 years, 4 years or 5 years ago, or any amount of time in between.

The compounds described may be used in the treatment of sepsis in an animal. Sepsis is commonly defined as life-threatening organ dysfunction caused by a dysregulated host response to infection (JAMA. 2016 Feb. 23; 315(8): 801-810). Intestinal barrier dysfunction is thought to contribute to the development of multiple organ dysfunction syndrome in sepsis (Shock. 2016 July; 46(1): 52-59). Observational studies have showed evidence of intestinal hyperpermeability in critically ill patients in general (Semin Respir Crit Care Med. 2011; 32:626-638) and specifically in septic patients (Biochem Med (Zagreb) 2013; 23:107-111). Accordingly, compounds of the invention may be useful in treating sepsis, by strengthening the epithelial barrier and controlling intestinal permeability.

The compounds described may be used in the treatment of bacteremia in an animal. Bacteremia is the presence of bacteria in the blood. It can be caused in patients with weakened epithelial barrier function (for example, patients that have undergone cancer treatment or organ transplantation), wherein pathogens from the GI tract can cross into the bloodstream. Accordingly, compounds of the invention may be useful in treating sepsis, by strengthening the epithelial barrier and controlling intestinal permeability.

The compounds described may be used in the treatment of mucositis in an animal. Mucositis is the painful inflammation and ulceration of the mucous membranes lining the digestive tract, usually as an adverse effect of chemotherapy and radiotherapy treatment for cancer (Ridge J A, Glisson B S, Lango M N, et al. “Head and Neck Tumors” in Pazdur R, Wagman L D, Camphausen K A, Hoskins W J (Eds) Cancer Management: A Multidisciplinary Approach. 11 ed. 2008.) Accordingly, compounds of the invention may be useful in treating mucositis, by repairing the mucous membranes lining the digestive tract.

The compounds described may be used in the treatment of microbiome dysbiosis in an animal. Dysbiosis is characterized by a decrease in microbial diversity and increase in proinflammatory species. This imbalanced microbiota is unable to protect from pathogenic organisms, that can trigger inflammation and produce genotoxins or carcinogenic metabolites (Precision Medicine for Investigators, Practitioners and Providers, 2020). It can be brought on by direct-acting antimicrobials. In combination with decreased epithelial barrier function, it can lead to further diseases described in, by way of translocation of the dysbiotic microbiota across the epithelial barrier. Accordingly, compounds of the invention may be useful in treating mucositis, by repairing the mucous membranes lining the digestive tract, and causing restoration of normal function of gut microbiota.

Further diseases that may be treated by compounds of the invention include Atopic Dermatitis, Asthma, Allergic Rhinitis, Chronic rhinosinusitis, Eosinophilic Esophagitis, Meningitis, COPD, Periodontitis Bronchitis, Eczema, Inflammatory Bowel Disease, Coeliac Disease, Leaky Gut Syndrome, Alzheimer Disease, Parkinson Disease, Chronic Depression, Autism, Diabetes, Obesity, Non-Alcoholic Steatohepatitis, Autoimmune Hepatitis, Liver Cirrhosis, Rheumatoid Arthritis, Multiple Sclerosis, Systemic Lupus Erythematosus, Ankylosing Spondylitis, and Intestinal Tissue Inflammation.

The compounds of the invention may also find use in travel medicine, and in treating diseases associated with travel. Traveller's diseases include diarrhoea (e.g. traveller's diarrhoea) which is a digestive tract disorder that commonly causes loose stools and abdominal cramps. It's caused by eating contaminated food or drinking contaminated water. Bacteria are the most common cause of traveller's diarrhoea. The most common pathogens identified are enterotoxigenic Escherichia coli, followed by Campylobacter jejuni, Shigella spp., and Salmonella spp. These bacteria infiltrate the epithelial lining and cause the inflammatory reaction, which is manifested by the diarrhoea. Although discouraged by doctors, antibiotics are very commonly used to alleviate this affliction. Side effects of antibiotic use is emergence of AMR bacteria and dysbiosis. The most common antibiotic used is Rifaximin, which is a poorly absorbed antibiotic that is not distributed systemically. Rifaximin reduces the symptoms of enteric infection, often without eradicating the pathogen. There is a scarcity of prophylactic treatments available to prevent the bacterial infiltration of the epithelial lining by these pathogens. In some embodiments, the compounds of the invention may therefore be used as prophylactics that can be ingested before exposure to pathogens that cause travellers diarrhoea, thereby preventing infection.

Other diseases that may be treated by compounds of the invention include Non alcoholic fatty liver disease and Necrotizing Enterocolitis.

The compounds of the invention are particularly useful against infections of bacterial strains that are tolerant against conventional antibiotics. Bacterial species include, but are not limited to, Yersenia, Salmonella, Shigella, Campylobacter, Clostridium, Heliobacter, Mycobacterium, Pseudomonas, Haemophilus, Moraxella, Escherichia, Neisseria and Staphyllococcus strains. Also embraced is the targeting of viruses, including HIV, RSV, herpes, hepatitis and influenza viruses, which are also believed to be a target for the antimicrobial peptides stimulated by the present invention. The viruses may be DNA or RNA viruses.

In some preferred embodiments, the compounds of the invention may be used to treat HIV infections. In some preferred embodiments, the compounds of the invention may be used to treat AIDS. In some preferred embodiments, the compounds of the invention may be used in the long-term treatment of AIDS recurring patients, particularly those who may die prematurely from epithelial barrier dysfunction. In some preferred embodiments, the compounds of the invention may be used to treat a patient diagnosed with HIV infection and/or AIDS, who additionally suffers from gut epithelial barrier dysfunction or a condition or disease caused by gut epithelial barrier dysfunction, or a condition or disease that may be treated by improvement or restoration of epithelial barrier function. In some embodiments, compounds of the invention may be used to treat a patient with antiretroviral therapy (ART)-suppressed HIV infection and a history of AIDS. The patient may be suffering from AIDs, or may have been previously diagnosed with and/or treated for AIDs. In some embodiments, compounds of the invention may treat premature aging, and improve morbidity and mortality in HIV patients who have been treated with ART.

The compounds of the invention are also particularly useful against or in preventing microbial infections caused by Klebsiella pneumonia, Escherichia coli, Enterobacter spp., Serratia spp., Proteus spp., Providencia spp., Morganella spp., Enterococcus faecium, Staphylococcus aureus, Helicobacter pylori, Acinetobacter baumannii, Pseudomonas aeruginosa, Campylobacter (e.g. Campylobacter jejuni), Salmonella spp., Neisseria gonorrhoeae, Streptococcus pneumoniae, Haemophilus influenzae, Shigella spp. In some embodiments, the compounds are effective against or in preventing microbial infections caused by Nairovirus, Marburg Virus, Ebola virus, Coronaviridae, Mammarenavirus, Henipavirus, Phlebovirus, Chikungunya, Alphavirus (Togavirus), Zika, and Dengue and other Flavivirus.

Infections, conditions and diseases treatable according to the present invention also include, but are not limited to:
Shigellosis, endemic diarrhoea, dysentery, viral gastroenteritis, parasitic enteritis, Crohn's disease, ulcerative colitis, irritable bowel syndrome, precancerous states of the gastrointestinal tract, cancer of the gastrointestinal tract, diverticulitis, post-antibiotic diarrhoea, Clostridium difficile colitis, lactose intolerance, flatulence, gastritis, esophagitis, heartburn, gastric ulcer, ulcers associated with Helicobacter pylori, duodenal ulcer, short bowel syndrome, dumping syndrome, gluten enteropathy;
Eye infections optionally selected from conjunctivitis, stye, blepharitis, cellulitis of the eye, keratitis, corneal ulcer, trachoma, uveitis, canaliculitis and dacryocystitis;
Urinary tract and genital infections optionally selected from pyelonephritis, cystitis, gonorrhoea and urethritis;
Infections of the respiratory system optionally selected from bronchitis, pneumonia, rhinosinusitis, sinusitis, pharyngitis/tonsillitis, laryngitis and influenza; tuberculosis;
Skin infections optionally selected from boils, carbuncles, furuncles, cellulitis, abscesses, impetigo, and erysipelas.

In some embodiments, the compounds of the invention are effective against bacterial strains that are resistant to direct-acting antibiotics, i.e. antibiotics that exert their effect by direct interaction with the microbial species.

Compounds of the invention may also be HDAC inhibitors (“HDACi”). In some embodiments, the compound is a selective HDACi. In some preferred embodiments, the compound is a class 1 selective HDACi. In some embodiments, the compound is a class 2 selective HDACi. In some embodiments, the compound is a class 4 selective HDACi. In some embodiments, the compound is a selective HDACi for any one of HDAC1, HDAC2, HDAC3, HDAC8, HDAC4, HDAC5, HDAC6, HDAC7, HDAC9, HDAC10 and HDAC11. The compound may also be a class 3 selective HDACi, a class 2A selective HDACi, or a class 2B HDACi.

In some embodiments, the compound is a selective HDACi for HDAC1. In some embodiments, the compound is a selective HDACi for HDAC2. In some embodiments, the compound is a selective HDACi for HDAC3. In some embodiments, the compound is a selective HDACi for HDAC1 and HDAC2. In some embodiments, the compound is a selective HDACi for HDAC1 and HDAC3. In some embodiments, the compound is a selective HDACi for HDAC2 and HDAC3. In some embodiments, the compound is a selective HDACi for HDAC1, HDAC2 and HDAC3.

In some embodiments, the compound inhibits a HDAC described above with an IC50 of less than 500 μM, less than 450 μM, less than 400 μM, less than 350 μM, less than 300 μM, less than 250 μM, less than 200 μM, less than 150 μM, less than 100 μM, less than 50 μM, less than 45 μM, less than 40 μM, less than 35 μM, less than 30 μM, less than 25 μM, less than 20 μM, less than 15 μM, less than 10 μM, less than 9 μM, less than 8 μM, less than 7 μM, less than 6 μM, less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits a class 1 HDAC with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC1 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC2 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC3 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC1 and HDAC2 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC1 and HDAC3 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC2 and HDAC3 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

In some embodiments, the compound inhibits HDAC1, HDAC2 and HDAC3 with an IC50 of less than 5 μM, less than 4 μM, less than 3 μM, less than 2 μM, less than 1.5 μM, less than 1.4 μM, less than 1.3 μM, less than 1.2 μM, less than 1.1 μM, less than 1.0 μM, less than 0.9 μM, less than 0.8 μM, less than 0.7 μM, less than 0.6 μM, less than 0.5 μM, less than 0.4 μM, less than 0.3 μM, less than 0.2 μM or less than 0.1 μM.

The IC50 may be measured by in-vitro enzymatic assay, such as that described in the Examples.

In some embodiments, compounds of the invention may therefore be useful in the treatment of diseases that are ameliorated by the inhibition of HDAC activity, such as cancer and cardiovascular diseases.

In some embodiments, compounds of the invention may be useful in the treatment of acute lung injury. In some embodiments, compounds of the invention may be useful in the treatment of acute respiratory distress syndrome. In some embodiments, compounds of the invention may be useful in the treatment of respiratory fever. In some embodiments, compounds of the invention may be useful in the treatment of sepsis induced-acute lung injury. In some embodiments, compounds of the invention may be useful in the treatment of sepsis induced-acute respiratory distress syndrome. In some embodiments, compounds of the invention may be useful in the treatment of sepsis induced-respiratory failure. Sepsis—or otherwise—induced acute lung injury may deteriorate into acute respiratory distress syndrome and respiratory failure, in some instances. In some particularly preferred embodiments, a compound of the invention is a HDAC3 inhibitor for use in the treatment of these diseases.

### Methods of Administration

The agents (i.e. the compound described here, plus one or more other agents) may be formulated together in a single dosage form, or alternatively, the individual agents may be formulated separately and presented together in the form of a kit, optionally with instructions for their use.

For example, the compounds described herein may in any aspect and embodiment also be used in combination therapies, e.g. in conjunction with other agents. Such agents may be as follows:

### Butyrate and PBA

Sodium phenylbutyrate is a known medicament. For example it has been marketed by Ucyclyd Pharma (Hunt Valley, USA) under the trade name Buphenyl and by Swedish Orphan Biovitrum AB (Sweden) as Ammonaps. It has been used to treat urea cycle disorders (Batshaw et al. (2001) J. Pediatr. 138 (1 Suppl): S46-54; discussion S54-5). Scandinavian Formulas, Inc. Sellersville, PA supplies sodium phenylbutyrate worldwide for clinical trials. Sodium phenylbutyrate is also under investigation for the treatment of some sickle-cell disorders (Blood Products Plasma Expanders and Haemostatics) and for use as a potential differentiation-inducing agent in malignant glioma and acute myeloid leukaemia. It has also been investigated in respect of cystic fibrosis pathology due to its capacity to traffic DeltaF508-cystic fibrosis transmembrane conductance regulator (CFTR) to the cell membrane and restore CFTR chloride function at the plasma membrane of CF lung cells in vitro and in vivo (Roque et al. J Pharmacol Exp Ther. 2008 September; 326(3):949-56. Epub 2008 Jun. 23). It is believed in the literature that phenylbutyrate is a prodrug which is metabolized in the body by beta-oxidation to phenylacetate.

### Vitamin D

The new class of compounds works synergistically with vitamin D as shown with pyridin-3-ylmethyl (4-((2-aminophenyl)carbamoyl)benzyl)carbamate. Vitamin D type compounds are discussed in US20080038374 or WO/2008/073174. Where the term “Vitamin D” is used herein, it is used in a broad sense to encompass Vitamin D3 (or “1,25 D3”) and its hormonally active forms, to include compounds which are structurally similar to vitamin D3. Many of these compounds are recognized and comprise a large number of natural precursors, metabolites, as well as synthetic analogs of the hormonally active 1, 25-dihydroxyvitamin D3 (1α25 (OH)₂D3). This language is intended to include vitamin D3, or an analog thereof, at any stage of its metabolism, as well as mixtures of different metabolic forms of vitamin D3 or analogs thereof.

### Antibiotics

The compounds of the invention are particularly useful against infections of bacterial strains that are tolerant against conventional antibiotics. Nevertheless use of the compounds described herein in conjunction with conventional antibiotics, especially narrow spectrum antibiotics, may be preferred and forms one part of the present invention.

Example antibiotics include Penicillins, Penicillin G, Phenoxymethyl-penicillin, Flucloxacillin, Amoxycillin, Metronidazole, Cefuroxime, Augmentin, Pivmecillinam, Acetomycin, Ciprofloxacin and Erythromycin. Where these specific antibiotics are named, it will be appreciated that commonly available analogs may be used. As noted above, in certain aspects, it may be preferred to use the compounds described herein in conjunction with a known antibiotic, as follows:
(1) acute administration to the patient of an antibiotic for preferably 1, or 2, days with or without a compound of formula (I) or (Ia); followed by,
(2) administration to the patient of an effective amount of a compound of formula (I) or (la) for a further 2, 3, 4, 5 or more days.

Such a regime may have benefits in minimising the development of antibiotic resistance in the pathogen to be targeted.

### Isoleucine and Related Compounds

The amino acid L-isoleucine upregulates β-defensins expression in epithelial cells of cows (18). US2002-0076393 (Fehlbaum et al.); US2003-0109582 (Zasloff); U.S. Pat. No. 7,311,925 (Zasloff) also relate to the use of isoleucine, an active isomer thereof, and an active analog thereof, in each case for stimulation of the innate antimicrobial defence system.

The disclosure of all these references, in respect of these compounds, their definition, and their provision, is hereby specifically incorporated herein by cross-reference.

Also provided are pharmaceutical compositions comprising, in addition to one or more of the compounds of the invention, vitamin D or one of the other aforementioned compounds as a further ingredient. Such compositions can be formulated in any of the above mentioned formulations and dosage forms.

Oral or inhalation dosage forms are preferred, as described below.

### Dosages

In particular aspects of the invention there are provided methods for treating, preventing or counteracting the diseases described herein in a patient in need of the same, by administering to the patient an effective amount of a compound of the invention as described herein.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

Thus an effective amount in the present context would be one which is sufficient to demonstrate improvement or restoration of epithelial barrier function and/or reduction or prevention of bacterial translocation, or in achieving a clinical end-point for the relevant disease. In some cases, the effective amount would be one which is sufficient to demonstrate antimicrobial activity in vivo e.g. by stimulating (e.g. suppressing or counteracting down-regulation caused by several pathogens) synthesis of the cathelicidin LL-37 or other naturally occurring antibiotic peptide or protein e.g. a defensin. Stimulation may be towards, equal to, or above basal levels (i.e. normal levels in the absence of the infection and/or normal levels of epithelial barrier function).

By the term “antimicrobial activity” as used herein, is meant the ability to inhibit the growth of or actually kill a population of microbes which can be bacteria, viruses, protozoa or fungal microbes. Thus “antimicrobial activity” should be construed to mean both microbistatic as well as microbicidal activities. Antimicrobial activity should also be construed to include a compound which is capable of inhibiting infections, i.e. disease-causing capacity of microbes. Generally the use of the present invention will be such as to lead to secretion of the relevant peptide onto an epithelial surface.

Preferred dosages and dosage forms are described in more detail below.

A preferred dosage of a compound of type I may be: between 25 μg and 2000 mg; more preferably 0.05 mg to 500 mg; more preferably 0.1 to 250 mg; more preferably about 0.2 to 100 mg; more preferably less than or equal to about 50 mg/day.

Another preferred dosage of a compound of the invention is a dose of at least 5 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg.

Another preferred dosage of a compound of the invention is a dose of at least 50 mg/kg.

Another preferred dosage of a compound of the invention is a dose of less than 200 mg/kg, less than 190 mg/kg, less than 180 mg/kg, less than 170 mg/kg, less than 160 mg/kg, less than 150 mg/kg, less than 140 mg/kg, less than 130 mg/kg, less than 120 mg/kg, or less than 110 mg/kg.

Further preferred dosages of the invention are ranges comprising a start point of at least 5 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg, and an end point of less than 200 mg/kg, less than 190 mg/kg, less than 180 mg/kg, less than 170 mg/kg, less than 160 mg/kg, less than 150 mg/kg, less than 140 mg/kg, less than 130 mg/kg, less than 120 mg/kg, or less than 110 mg/kg.

In each case dosages can be split into 1, 2, 3, 4, 5, 6 or 7 doses per week or 1, 2 or 3 doses per day. For example 1, 2 or 3×3, 5 or 10 mg/week, 1 or 2×5 or 50 mg/day or 2×250 mg/week and so on. A preferred regime is less than 3×per day e.g. 1 or 3×3 mg/week, 2×1 mg/day or 2×5 mg/week.

A preferred dosage may be between 0.1 mg and 100 mg; between 0.2 mg and 50 mg; between 0.2 mg and 20 mg; optionally with vitamin D3. Dosages for Vitamin D may be of the order of 1000-10 000 IU daily.

Thus preferred dosages can be split into 1, 2, 3, or 4 doses per week or 1, 2 doses per day. For example 1 or 3×2 mg/week, 2×0.5 mg/day and so on.

Corresponding preferred weight\molar amounts for other compounds of the invention can be calculated by those skilled in the art based on the disclosure herein.

Each of the amounts disclosed herein and above may be administered as a weekly dosage or a daily dosage. Each weekly dosage may be split into doses given 1, 2 or 3 times, or more times. Each daily dosage may be split into doses given 1, 2 or 3 times, or more times. The dosage schedules disclosed herein and above may comprise or consistent of 1, 2, 3, 4, 5, 6 7 total days dosing, or 1, 2, 3, 4, 5, 6, 7, 8, 9, 10, 15, 20, 25, 30, 35, 40, 45, 50 or 55 total weeks dosing.

### Dosage Forms

The compound of the invention is preferably administered in an oral dosage form such as, but not limited to, a tablet, a capsule, a solution, an emulsion, a suspension, a powder, a paste, an elixir, and a syrup. Administration of the compounds can also include incorporation into nanoparticles or ultrafine particle with lengths in two or three dimensions greater than 0.001 micrometer (1 nanometer) and smaller than about 0.1 micrometer (100 nanometers) composed of organic and inorganic biocompatible materials.

Compositions for oral use may be in the form of hard gelatin capsules in which the active ingredient is mixed with an inert solid diluent, such as but not limited to cellulose, hydroxypropylmethyl cellulose, calcium carbonate, calcium phosphate or kaolin, or as soft gelatin capsules in which the active ingredient is mixed with an oil such as but not limited to arachidonic oil, glycerides, peanut oil, liquid paraffin, or olive oil.

Suitable pharmaceutically acceptable excipients for a tablet formulation include, for example, inert diluents such as but not limited to lactose, sodium carbonate, calcium phosphate or calcium carbonate, granulating and disintegrating agents such as but not limited to corn starch or algenic acid; binding agents such as but not limited to starch; lubricating agents such as but not limited to magnesium stearate, stearic acid or talc; preservative agents such as but not limited to ethyl or propyl p-hydroxybenzoate, and anti-oxidants, such as but not limited to ascorbic acid. The tablets may be uncoated or coated either to modify their disintegration and the subsequent absorption of the active ingredient within the gastrointestinal tract, or to improve their stability and/or appearance, in either case, using conventional coating agents and procedures well known in the art, enterosoluble coating agents with various resistance to the pH and enzymes in the gut.

Other administration forms are also useful, these include but not are limited to topical administration forms, which are in particular useful against infections of the skin, these include for example creams, oils, lotions, and ointments. Yet further dosage forms include dosage forms for delivery to the respiratory system including the lungs, such as aerosols and nasal spray devices or by rectal enema.

In some particularly preferred embodiments, compounds of the invention are administered as a lozenge. Lozenges or troches are pharmaceutical dosage forms which may be particularly effective in treating conditions that affect the tissues and epithelial barriers contained within the oral cavity and throat. A typical lozenge or troche is composed predominantly of an inert vehicle, carrier, or diluent. A compound of the invention is interspersed within this carrier. The lozenge will slowly dissolve when placed in the oral cavity thereby releasing the medicinal agent so that it may come in contact with the tissues of the mouth and throat. The term “lozenge” as used herein embraces dosage forms where the product is formed by cooling a sugar-based or sugar alcohol based (e.g. isomalt) molten mass containing a compound of the invention Suitably, the term “molten lozenge-forming composition” embraces a sugar-based or sugar alcohol based (e.g. isomalt) molten mass. The lozenge is a solid dosage form which is intended to be sucked by a patient.

### Compounds of the Invention

The compounds of the invention to those capable of ameliorating barrier function or block microbial translocation, and may also induce antimicrobial peptides (AMPs) of the innate immune system.

Compounds of the invention are defined according to formula I or Ia as described herein. Certain preferred embodiments of the compounds are now described.

### Group Q

In the compounds of formula (I) or (Ia), the group Q is selected from Q1, Q2, Q3, Q4, Q5 and Q6:

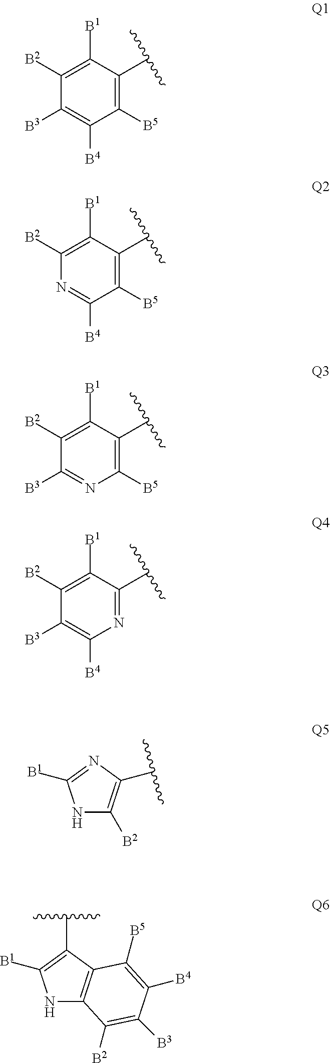

wherein one of B¹ and B² (and B³, B⁴ and B⁵ where present) is a group of formula —X—R^{X} and the others are independently selected from H and R^{B}. In other words, each possible Q group includes one —X—R^{X} substituent in an available position, the other available positions being either unsubstituted (—H) or substituted with a group —R^{B}.

In some preferred embodiments, Q is a phenyl group, Q1:

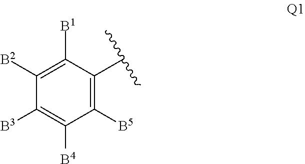

wherein one of B¹, B², B³, B⁴ and B⁵ is a group of formula —X—R^{X} and the others are independently selected from H and R^{B}.

In some embodiments, B³ is a group of formula —X—R^{X} and B¹, B², B⁴ and B⁵ are independently selected from H and R^{B}. In some embodiments, B³ is a group of formula —X—R^{X} and B¹, B², B⁴ and B⁵ are H. Accordingly, in these embodiments, Q is a group of formula:

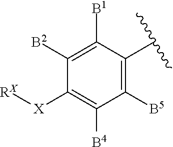

In other embodiments, B² is a group of formula —X—R^{X} and B¹, B³, B⁴ and B⁵ are independently selected from H and R^{B}. In still further embodiments, B¹ is a group of formula —X—R^{X} and B², B³, B⁴ and B⁵ are independently selected from H and R^{B}.

In some embodiments, one of B¹, B², B³, B⁴ and B⁵ is a group of formula —X—R^{X} and the others are independently H.

In some embodiments B² or B³ is a group of formula —X—R^{X} and the others are independently H.

In some embodiments B³ is a group of formula —X—R^{X} and the others are independently H.

In some embodiments, Q is a pyridyl group Q2, Q3 or Q4:

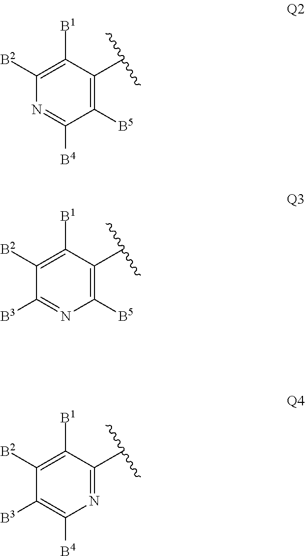

wherein one of B¹, B², B³ and B⁴ is a group of formula —X—R^{X} and the others are independently selected from H and R^{B}.

In some embodiments, one of B¹, B², B³ and B⁴ is a group of formula —X—R^{X} and the others are independently H. In some of said embodiments X is a covalent bond and R^{X} is —H.

In some embodiments, Q is Q2.

In some embodiments, Q is Q3.

In some embodiments, Q is Q4.

In some embodiments, 0 is an imidazolyl group Q5:

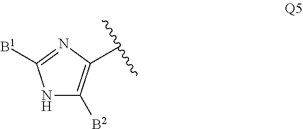

wherein one of B¹ and B² is a group of formula —X—R^{X} and the other is selected from H and R^{B}.

In some embodiments, one of B¹ and B² is a group of formula —X—R^{X} and the other is —H.

In some of said embodiments X is a covalent bond and R^{X} is —H.

In some embodiments 0 is an indolyl group Q6:

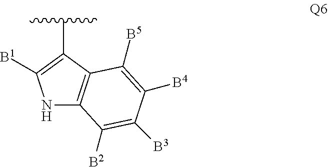

wherein one of B¹, B², B³, B⁴ and B⁵ is a group of formula —X—R^{X} and the others are independently selected from H and R^{B}.

In some embodiments, one of B¹, B², B³, B⁴ and B⁴ is a group of formula —X—R^{X} and the others are independently H. In some of said embodiments X is a covalent bond and R^{X} is —H.

### Group R^{B}

Where present, each —R^{B} group is independently selected from halogen, —CF₃, —R, —OH, —OR, —OCF₃, —C(═O)OH, —C(═O)OR, —C(═O)R, —OC(═O)R, —NH₂, —NHR, —NR₂, —NO₂, —C(═O)NH₂, —C(═O)NHR, C(═O)NR₂, —S(═O)R, —S(═O)₂R, —S(═O)₂NR₂, or —CN.

In some embodiments, —R^{B} is selected from halogen (i.e. —F, —Cl, —Br, —I), —CF₃, —R, —OH, —OR, —NH₂, —NHR, —NR₂, —NO₂, and —CN.

In some embodiments, —R^{B} is selected from —OH, —OR, —NH₂, —NHR, and —NR₂.

In some embodiments, —R^{B} is selected from —OH or —OR.

In some embodiments. —R^{B} is —OR.

In some embodiments. —R^{B} is —OMe.

In some embodiments, —R^{B} is —R.

In some embodiments. —R^{B} is -Me.

### Group X—R^{X}

In the group —X—R^{X}, X is selected from a covalent bond or C₁₋₃alkylene and R^{X} is selected from H, R^{XX} or R^{XY}.

In some embodiments, X is a covalent bond (i.e. the group —X—R^{X} is a group of formula —R^{X}).

In some embodiments, X is selected from C₁₋₃alkylene.

In some embodiments, X is selected from —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments, X is —CH₂—.

In some embodiments R^{X} is H.

In some embodiments, R^{X} is R^{XX}, R^{XX} is selected from halogen, —CF₃, —OH, —OR, —OCF₃, —C(═O)OH, —NO₂, —NH₂, —NHR, —NR₂, —C(═O)NH₂, —C(═O)NR₂, —S(═O)R, —S(═O)₂R, —S(═O)₂NR₂, and —CN.

In some embodiments, R^{XX} is selected from —OH, —OR, —NO₂, —NH₂, NHR, and NR₂.

In some embodiments, R^{XX} is selected from —OR, —NO₂ and —NR₂.

In some embodiments, R^{XX} is —OMe, —NO₂, or —NMe₂.

In some embodiments, R^{X} is R^{XY} wherein R^{XY} is a group of formula -L^{X}-R^{YY}.

L^{X} is selected from —NH—C(═O)—O—, —NH—C(═O)—NH—, —NH—C(═O)—, —NR—C(═O)—, —O—C(═O)—NH—, —O—C(═O)—O—, —O—(C=O)—, —C(═O)—NH—, —C(═O)—O—, and —C(═O)—.

In some embodiments, L^{X} is selected from —NH—C(═O)—O—, —NH—C(═O)—, and —C(═O)—NH—.

In some embodiments, L^{X} is selected from —NH—C(═O)—O—, —NH—C(═O)—, NR—C(═O)—, or —O—(C=O)—. In some embodiments, L^{X} is selected from —NH—C(═O)— or —NH—C(═O)—O—.

In some embodiments, L^{X} is —NH—C(═O)—O—. In some embodiments, L^{X} is —NH—C(═O)—. In some embodiments, L^{X} is NR—C(═O)—. In some embodiments, L^{X} is —O—(C=O)—. In some embodiments, L^{X} is —C(═O)—NH—.

R^{YY} is selected from C₁₋₄alkyl, C₃₋₆cycloalkyl, —C₆₋₁₄aryl, -L^{Y}-C₆₋₄aryl, -L^{Y}-O—C₆₋₁₄aryl —C₅₋₆heteroaryl, -L^{Y}-C₅₋₆heteroaryl, -L^{Y}-C₅₋₆heteroaryl, -L^{Y}-O—C₅₋₆heteroaryl, L^{Y}-O-L^{Y}-C≡N, L^{Y}-O-L^{Y}-C=R, L^{Y}-O-L^{Y}-C≡CH, -L^{Y}-O-L^{Y}-NR^{N}—C(═O)—R, L^{Y}-O-L^{Y}-NH—C(═O)—R, L^{Y}-O-L^{Y}-NR—C(═O)—R, L^{Y}-C≡N, L^{Y}-C=R, and L^{Y}-C≡CH, wherein -L^{Y}- is C₁₋₃alkylene and wherein each of said R groups is optionally substituted.

In some embodiments, R^{YY} is independently: —C₆₋₁₄aryl, -L^{Y}-C₆₋₁₄aryl, —C₅₋₆-heteroaryl, or -L^{Y}-C₅₋₆heteroaryl, wherein said C₆₋₁₄aryl and C₅₋₆heteroaryl groups are optionally substituted.

In some embodiments, R^{YY} is independently: -Ph, -L^{Y}-Ph, C₅₋₆heteroaryl, or -L^{Y}-C₅₋₆heteroaryl, wherein said Ph and C₅₋₆heteroaryl groups are optionally substituted.

In some embodiments, R^{YY} is independently: -L^{Y}-Ph or -L^{Y}-C₅₋₆heteroaryl, wherein said Ph and C₅₋₆heteroaryl groups are optionally substituted.

In some embodiments, R^{YY} is independently-L^{Y}-C₆₋₁₄aryl, wherein said C₆₋₁₄aryl is optionally substituted.

In some embodiments, R^{YY} is independently: -L^{Y}-Ph, wherein said Ph is optionally substituted.

In some embodiments, R^{YY} is independently: -L^{Y}-C₅₋₆heteroaryl, L^{Y}-O-L^{Y}-C≡CH, L^{Y}-O-L^{Y}-NH—C(═O)—R, L^{Y}-C=R, L^{Y}-C≡CH.

In some embodiments, -L^{Y}- is independently selected from —CH₂—, —CH₂CH₂—, —CH₂CH₂CH₂—, —CH(CH₃)—, —CH(CH₃)CH₂—, —CH₂CH(CH₃)—, and —CH(CH₂CH₃)—.

In some embodiments, -L^{Y}- is independently —CH₂—.

In some embodiments each of said R^{YY} groups is optionally substituted with one or more substituents selected from: —F, —Cl, —Br, —I, —R, —CF₃, —OH, —OR, —OCF₃, —NO₂, -L^{YY}-OH, -L^{YY}-OR, —NH₂, —NHR, —NR₂, -L^{YY}-NH₂, -L^{YY}-NHR, -L^{YY}-NR₂, —CO₂H, —CO₂R, -L^{YY}-CO₂H, -L^{YY}-CO₂R, -Ph, and -L^{YY}-Ph-, wherein L^{YY} is C₁₋₃alkylene.

In some embodiments, each of said R^{YY} groups is optionally substituted with one or more substituents selected from: —OH, —OR, -L^{YY}-OH, -L^{YY}-OR, —NH₂, —NHR, —NR₂, -L^{YY}-NH₂, -L^{YY}-NHR, -L^{YY}-NR₂, -L^{YY}-CO₂H, -L^{YY}-CO₂R, -Ph, and -L^{YY}-Ph-, wherein L^{YY} is C₁₋₃ alkylene.

In some embodiments, each of said R^{YY} groups is optionally substituted with one or more substituents selected from —NH₂, —NHR, —NR₂, -L^{YY}-CO₂H, and -L^{YY}-CO₂R, wherein L^{YY} is C₁₋₃alkylene.

In some embodiments, R^{YY} is independently: -L^{Y}-Ph, wherein said Ph is substituted with one or more substituents selected from: —OH, —OR, -L^{YY}-OH, -L^{YY}-OR, —NH₂, —NHR, —NR₂, -L^{YY}-NH₂, -L^{YY}-NHR, -L^{YY}-NR₂, -L^{YY}-CO₂H, -L^{YY}-CO₂R, -Ph, and -L^{YY}-Ph-, wherein L^{YY} is C₁₋₃alkylene.

In some embodiments, R^{YY} is independently: -L^{Y}-Ph, wherein said Ph is substituted with one or more substituents selected from: —NH₂, —NHR, and —NR₂.

In some embodiments, R^{YY} is independently: —C₅₋₆heteroaryl, wherein said C₅₋₆heteroaryl is substituted with one or more substituents selected from: —OH, —OR, -L^{YY}-OH, -L^{YY}-OR, —NH₂, —NHR, —NR₂, -L^{YY}-NH₂, -L^{YY}-NHR, -L^{YY}-NR₂, -L^{YY}-CO₂H, -L^{YY}-CO₂R, -Ph, and -L^{YY}-Ph-, wherein L^{YY} is C₁₋₃alkylene.

In some embodiments, R^{YY} is independently: —C₅₋₆heteroaryl, wherein said C₅₋₆heteroaryl is substituted with -L^{YY}-CO₂R.

In some embodiments, -L^{YY}- is independently selected from —CH₂—, —CH₂CH₂—, —CH₂CH₂CH₂—, —CH(CH₃)—, —CH(CH₃)CH₂—, —CH₂CH(CH₃)—, and —CH(CH₂CH₃)—.

In some embodiments, -L^{YY}- is independently —CH₂—.

### Linker L

In the compounds of formula (I) or (Ia), the linker group L is selected from —(CH₂)ₘ—, —C(═O)—, —(CH₂)ₘ—C(═O)—, —O—(CH₂)ₘ—C(═O)—, —O—C(═O)—(CH₂)ₘ—(C=O)—, —NH—C(═O)—, —NR—C(═O)—, —NH—(CH₂)ₘ—C(═O)—, —NR—(CH₂)ₘ—C(═O)—, —NH—C(═O)—(CH₂)ₘ—C(═O)—, —NR—C(═O)—(CH₂)ₘ—C(═O)—, —C(═O)—NH—(CH₂)ₘ—C(═O)—, and —(CH₂)ₘ—(CHR^{L})—C(═O)—, where m is an integer from 1 to 4.

In some embodiments, L is selected from —(CH₂)ₘ—, —C(═O)—, —(CH₂)ₘ—C(═O)—, —NH—C(═O)—, —NR—C(═O)—, —NH—C(═O)—(CH₂)ₘ—C(═O)—, —C(═O)—NH—(CH₂)ₘ—C(═O)—, and —(CH₂)ₘ—(CHR^{L})—C(═O)—.

In some embodiments, L is selected from —(CH₂)ₘ—, —C(═O)—, —NH—C(═O)—, and —NR—C(═O)—.

In some embodiments, L is —C(═O)—. Accordingly, the compound may be a compound of formula (II):

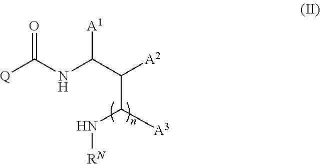

In some embodiments, L is —C(═O)—NH—(CH₂)ₘ—C(═O)—. Accordingly, the compound may be a compound of formula (III):

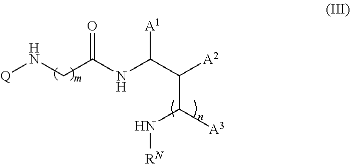

In some embodiments, L is —(CH₂)ₘ—(CHR^{L})—C(═O)—, wherein R^{L} is as defined herein. Accordingly, the compound may be a compound of formula (IV):

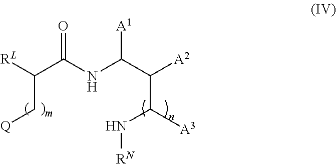

### Group R^{L}

Where present, e.g. in compounds of formula (Ic), the group R^{L} is selected from halogen, —R^{LL}, —CF₃, —OH, —OR^{LL}—, —NO₂, —NH₂, —NHR^{LL}, —NR₂, —NH—C(═O)—R^{LL}, —NH—C(═O)—O—R^{LL}.

In some embodiments, R^{L} is selected from —NH₂, —NHR^{LL}, —NH—C(═O)—R^{LL} and —NH—C(═O)—O—R^{LL}, wherein R^{LL} is as previously defined.

In some embodiments, R^{L} is NH₂.

In some embodiments, R^{L} is —NHR^{LL}.

In some embodiments, R^{L} is —NH—C(═O)—O—R^{LL}.

R^{LL} is selected from —C₁₋₄alkyl, —C₃₋₆cycloalkyl, -Ph, -L^{L}-Ph, —C₅₋₆heteroaryl, and -L^{L}-C₅₋₆heteroaryl wherein -L^{L}- is C₁₋₃alkylene, wherein said -Ph and —C₅₋₆heteroaryl are optionally substituted.

In some embodiments said -Ph and —C₅₋₆heteroaryl are optionally substituted with one or more groups selected from: —F, —Cl, —Br, —I, —R, —CF₃, —OH, —OR, —OCF₃, —NO₂, —NH₂, —NHR, —NR₂, —CO₂H, —CO₂R.

In some embodiments, R^{LL} is selected from -Ph, -L^{L}-Ph, —C₅₋₆heteroaryl, and -L^{L}-C₅₋₆heteroaryl.

In some embodiments, R^{LL} is selected from -L^{L}-Ph and -L^{L}-C₅₋₆heteroaryl.

In some embodiments, R^{LL} is -L^{L}-Ph.

In some embodiments, R^{LL} is —CH₂-Ph (-Bn).

L^{L} is selected from C₁₋₃ alkylene.

In some embodiments, L^{L} is selected from: —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

In some embodiments L^{L} is —CH₂— or —CH₂CH₂—.

In some embodiments L^{L} is —CH₂—.

### Group R^{N}

R^{N} is selected from H and optionally substituted C₁₋₄alkyl.

In some embodiments, R^{N} is H.

In some embodiments, R^{N} is C₁₋₄alkyl.

In some embodiments, when R^{N} is C₁₋₄alkyl, said C₁₋₄alkyl is optionally substituted with one or more substituents R^{N1}, wherein each R^{N1} is independently selected from halogen, —CF₃, —R, —OH, —OR, —OCF₃, —NH₂, —NHR, —NR₂, —NO₂ and —CN, wherein each R is independently C₁₋₄alkyl.

In some embodiments, R^{N1} is independently selected from —OH, —OR, —NH₂, —NHR, —NR₂.

In some embodiments, R^{N1} is OH or NH₂.

In some embodiments R^{N1} is NH₂.

In some embodiments, R^{N} is selected from methyl, ethyl, n-propyl, iso-propyl, n-butyl, iso-butyl, sec-butyl, and tert-butyl and is optionally substituted.

In some embodiments, R^{N} is ethyl and is optionally substituted.

In some embodiments, R^{N} is ethyl, substituted with at least one R^{N1}.

In some embodiments, R^{N} is —CH₂CH₂NH₂.

Groups A¹, A², A³

In some embodiments, A¹ and A², together with the atoms to which they are bound, form an optionally substituted C₆₋₁₄aryl group.

In some embodiments, said C₆₋₁₄aryl group is optionally substituted with one or more substituents R^{A2}, wherein each R^{A2} is independently selected from halogen, —CF₃, —R, —OH, —OR, —OCF₃, —NO₂, —C(═O)OH, —C(═O)OR, —C(═O)R, —OC(═O)R, —NH₂, —NHR, —NR₂, —C(═O)NH₂, —C(═O)NHR, —C(═O)NR₂, —S(═O)R, —S(═O)₂R, —S(═O)₂NR₂, and —CN.

In some embodiments, R^{A2} is independently selected from —R, —OH, —OR, —OCF₃, —NO₂, —NH₂, —NHR, —NR₂, and —CN.

In some embodiments, R^{A2} is independently —R.

In some embodiments, R^{A2} is independently methyl.

In some embodiments, A¹ and A², together with the atoms to which they are bound, form an optionally substituted phenyl group. Accordingly, the compound may be a compound of formula (V):

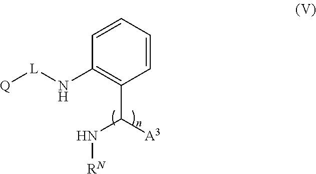

In some embodiments, A¹ and A², together with the atoms to which they are bound, form an optionally substituted naphthalene group. Accordingly, the compound may be a compound of formula (VI):

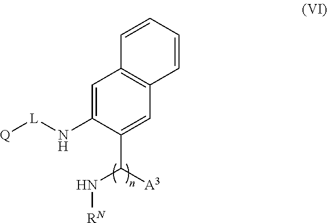

In the compounds of formula (V) and (VI) the phenyl and naphthalene rings may optionally be substituted with one or more substituents R^{A2} as defined above.

In some embodiments, A¹ and A², together with the atoms to which they are bound, form an unsubstituted phenyl or naphthalene group.

In some embodiments, A¹ and A², together with the atoms to which they are bound, form an unsubstituted phenyl group.

In some embodiments, A¹ and A², together with the atoms to which they are bound, form a phenyl substituted with one or more substituents R^{A2}.

In some embodiments, A¹ and A², together with the atoms to which they are bound, form a phenyl substituted with two substituents R^{A2}.

A³, if present, is selected from H and optionally substituted C₁₋₄alkyl.

In some embodiments, A³ is H.

In some embodiments, A³ is selected from methyl, ethyl, n-propyl, iso-propyl, n-butyl, iso-butyl, sec-butyl, and tert-butyl.

In some embodiments, A³ is methyl or ethyl.

In some embodiments, A³ is methyl.

n is selected from 0 and 1. When n is 0, A³ (and the atom to which it is attached) is absent. In some embodiments, n is 0. In some embodiments, n is 1.

Accordingly, the compound may be a compound of formula (VII):

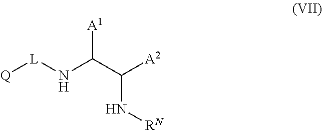

### Groups R

Each ‘R’, as used throughout these definitions, is independently a C₁₋₄ alkyl group.

In some embodiments, R is selected from methyl, ethyl, n-propyl, iso-propyl, n-butyl, iso-butyl, sec-butyl, and tert-butyl.

In some embodiments, R is methyl or ethyl.

In some embodiments, R is methyl.

### Certain Preferred Embodiments

In some embodiments:
A¹ and A², together with the atoms to which they are bound, form a phenyl ring, optionally substituted with one or more substituents R^{A2};
n is 0;
R^{N} is H.

Accordingly, the compound is a compound of formula (VIII):

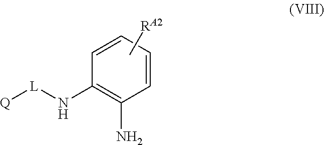

wherein Q, L and R^{A2} are as previously defined.

In some embodiments:
A¹ and A², together with the atoms to which they are bound, form a phenyl ring, optionally substituted with one or more substituents R^{A2};
n is 0;
R^{N} is H;
L is C(═O).

Accordingly, the compound is a compound of formula (IX):

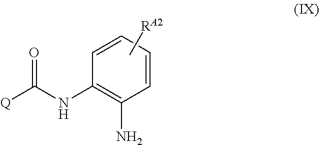

wherein Q and R^{A2} are as previously defined.

In some embodiments:
A¹ and A², together with the atoms to which they are bound, form a phenyl ring, optionally substituted with one or more substituents R^{A2};
n is 0;
R^{N} is H;
L is C(═O);
Q is Q1, wherein B³ is X—R^{X}.

Accordingly, the compound is a compound of formula (X):

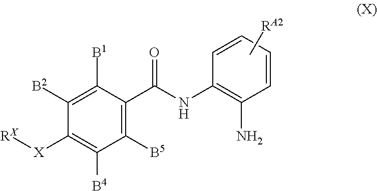

wherein X, R^{X}, B¹, B², B⁴, B⁵ and R^{A2} are as previously defined.

In some embodiments:
A¹ and A², together with the atoms to which they are bound, form a phenyl ring;
n is 0;
R^{N} is H;
L is C(═O);
Q is Q1, wherein B³ is X—R^{X} and B¹, B², B⁴ and B⁵ are all H.

Accordingly, the compound is a compound of formula (XI):

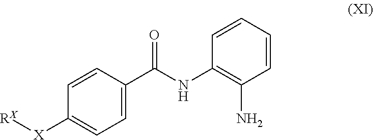

wherein X and R^{X} are as previously defined.

In some embodiments:
A¹ and A², together with the atoms to which they are bound, form a phenyl ring;
n is 0;
R^{N} is H;
Q is Q1.

Accordingly, the compound is a compound of formula (XII):

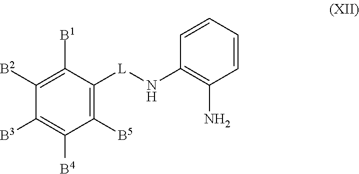

wherein B¹, B², B³, B⁴, B⁵ and L are as previously defined.

In some embodiments:
A¹ and A², together with the atoms to which they are bound, form a phenyl ring;
n is 0;
R^{N} is H;
Q is Q1, wherein B³ is X—R^{X} and B¹, B², B⁴ and B⁵ are all H.

Accordingly, the compound is a compound of formula (XIII):

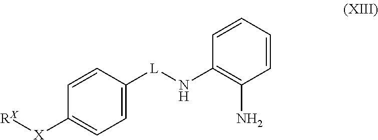

wherein X, R^{X}, and L are as previously defined.

### Specific Compounds

In some embodiments, the compound is selected from:

| # | Name | Structure |
|---|---|---|
| 1 | N-(2-aminophenyl)-4- {2-[(prop-2-yn-1- yl)oxy]acetamido} benzamide (compound 1) | 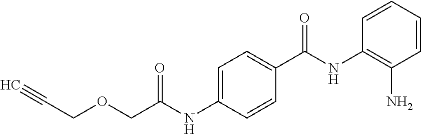 [SMILES: C#CCOCC(=O)Nc1ccc(C(=O)Nc2ccccc2N)cc1] |
| 2 | N-(2-aminophenyl)-4- [(methyl{[(prop-2-yn-1- yl)oxy]acetyl}amino)methyl] benzamide (compound 2.1) | 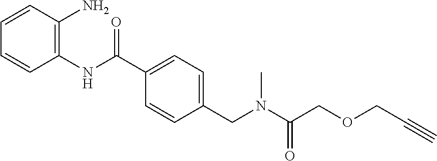 [SMILES: C#CCOCC(=O)N(C)Cc1ccc(C(=O)Nc2ccccc2N)cc1] |
| 3 | N-(2-aminophenyl)-4- {[N-methyl-2-(2- propanamidoethoxy)acetamido] methyl}benzamide (compound 2.2) | 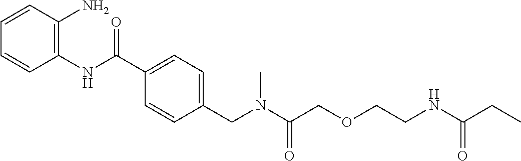 [SMILES: CCC(=O)NCCOCC(=O)N(C)Cc1ccc(C(=O)Nc2ccccc2N)cc1] |
| 4 | 4-[(2-aminophenyl) carbamoyl]phenyl hex-5- ynoate (compound 2.3) | 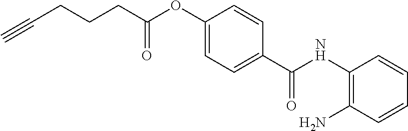 [SMILES: C#CCCCC(=O)Oc1ccc(C(=O)Nc2ccccc2N)cc1] |
| 5 | Pyridin-3-ylmethyl (4-((2-aminophenyl)- carbamoyl)benzyl)carbamate (Entinostat) | 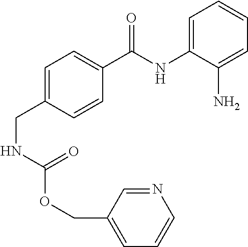 [SMILES: Nc1ccccc1NC(=O)c1ccc(CNC(=O)OCc2cccnc2)cc1] |

### Definitions

The term ‘alkyl’, as used herein, refers to a monovalent moiety obtained by removing a hydrogen atom from a saturated aliphatic hydrocarbon compound, preferably having from 1 to 4 carbon atoms (‘C₁₋₄alkyl’), which may be linear or branched.

Examples of C₁₋₄ alkyl groups include, but are not limited to, methyl, ethyl, n-propyl, iso-propyl, n-butyl, iso-butyl, sec-butyl, tert-butyl. In certain instances, methyl or ethyl groups may be preferred.

Similarly, the term ‘alkylene’ refers to a divalent moiety obtained by removing two hydrogen atoms from a saturated aliphatic hydrocarbon compound, preferably having from 1 to 3 carbon atoms (‘C₁₋₃alkylene’), which may be linear or branched.

Examples of C₁₋₃alkylene groups include, but are not limited to, —CH₂—, —CH₂CH₂—, —CH₂CH₂CH₂—, —CH(CH₃)—, —CH(CH₃)CH₂—, —CH₂CH(CH₃)—, or —CH(CH₂CH₃)—.

The term ‘cycloalkyl’, as used herein, refers to a monovalent moiety obtained by removing a hydrogen atom from a saturated alicyclic hydrocarbon compound, preferably having from 3 to 6 ring atoms (‘C₃₋₆cycloalkyl’).

Examples of C₃₋₆cycloalkyl groups include, but are not limited to, cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, dimethylcyclopentyl, methylcyclohexyl, dimethylcyclohexyl, cyclopropylmethyl and cyclohexylmethyl.

The term “C₆₋₁₄ aryl”, as used herein, pertains to a monovalent moiety obtained by removing a hydrogen atom from an aromatic ring atom of a C₆₋₁₄ aromatic compound, said compound having one ring, or two or more rings (e.g., fused), and having from 6 to 14 ring atoms, and wherein at least one of said ring(s) is an aromatic ring. Preferably, each ring has from 6 to 10 ring atoms. The term “C₆₋₁₄ aromatic ring” may also be used and should be construed accordingly; this may refer to a multivalent moiety.

The ring atoms may be all carbon atoms, as in “carboaryl groups”, in which case the group may conveniently be referred to as a “C₆₋₁₄ carboaryl” group.

Examples of C₆₋₁₄carboaryl groups include, but are not limited to, those derived from benzene (i.e. phenyl) (C₆), naphthalene (C₁₀), anthracene (C₁₄), and phenanthrene (C₁₄).

Examples of aryl groups which comprise fused rings, one of which is not an aromatic ring, include, but are not limited to, groups derived from indene and fluorene, e.g.:

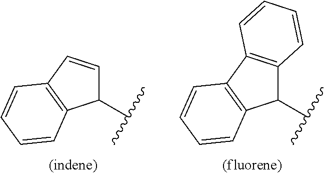 [SMILES: *C1C=Cc2ccccc21 ; *C1c2ccccc2-c2ccccc21]

The term ‘heteroaryl’, as used herein, refers to a monovalent moiety obtained by removing a hydrogen atom from an aromatic ring atom of a heteroaromatic compound, i.e. a compound containing at least one aromatic ring, wherein the ring atoms include at least one heteroatom. Possible heteroatoms include but are not limited to oxygen, nitrogen, and sulphur. Preferably, the aromatic ring has from 5 to 6 ring atoms, of which from 0 to 4 are ring heteroatoms. In this case, the group is referred to as a ‘C₅₋₆heteroaryl’ group, wherein ‘C₅₋₆’ denotes ring atoms whether carbon atoms or heteroatoms.

Examples of C₅₋₆heteroaryl group include, but are not limited to, C₅ heteroaryl groups derived from furan (oxole), thiophene (thiole), pyrrole (azole), imidazole (1,3-diazole), pyrazole (1,2-diazole), triazole (1,2,3-triazole, 1,2,4-triazole), oxazole, isoxazole, thiazole, isothiazole, oxadiazole, and oxatriazole; and C₆heteroaryl groups derived from isoxazine, pyridine (azine), pyridazine (1,2-diazine), pyrimidine (1,3-diazine; e.g., cytosine, thymine, uracil), pyrazine (1,4-diazine), triazine, tetrazole, and oxadiazole (furazan).

The term ‘halo’ or ‘halogen’ refers to —F, —Cl, —Br, and —I substituents. Fluoro (—F) and chloro (—Cl) substituents are usually preferred.

### Isomers, Salts, Solvates, and Protected Forms

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

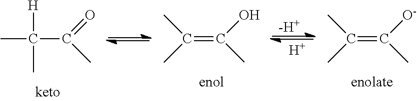

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

Unless otherwise specified, a reference to a particular compound also includes ionic, salt, solvate, and protected forms of thereof, for example, as discussed below.

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci., 66, 1-19 (1977).

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂+, NHR₃+, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t-butyl ether; a benzyl, benzhydryl (diphenylmethyl), or trityl (triphenylmethyl) ether; a trimethylsilyl or t-butyldimethylsilyl ether; or an acetyl ester (—OC(═O)CH₃, —OAc).

For example, an aldehyde or ketone group may be protected as an acetal or ketal, respectively, in which the carbonyl group (>C=O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases, as an N-oxide (>NO.).

For example, a carboxylic acid group may be protected as an ester for example, as: an C₁₋₇ alkyl ester (e.g. a methyl ester; a t-butyl ester); a C₁₋₇ haloalkyl ester (e.g., a C₁₋₇ trihaloalkyl ester); a triC₁₋₇ alkylsilyl-C₁₋₇alkyl ester; or a C₅₋₂₀aryl-C₁₋₇ alkyl ester (e.g. a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, in as much as it may be used by those skilled in the art to carry out the invention, is hereby specifically incorporated herein by cross-reference.

### FIGURES

FIG. 1: E. coli mouse infection model. CFU measured in tissue: colon, ileum, liver, spleen. Statistical analysis was performed by comparing all conditions to the vehicle-treated group and using one-way ANOVA with Dunnett post-hoc test, *** p<0.001, **** p<0.0001, ns non-significant.

FIG. 2: Induction test. Compounds 2.1, 2.2 and 2.3 induced expression of ProLL-37-luciferase fusion protein in HT-29 CampLuc MN8 reporter cell line after 24h. Corresponding concentrations of compound 1 (2-128 μM) and Entinostat (2.5 μM) were used as positive controls. The results are presented as an average of luminescence signal relative to control (untreated cells; value 1) from three independent experiments±SD. Statistical analysis was performed by comparison of each compound-treatment conditions to the corresponding vehicle control using two-way ANOVA with Dunnett post-hoc test. Only significant changes were indicated, ** p<0.01, *** p<0.001, **** p<0.0001. Entinostat treatment (Entino) was compared to control by using t-test, ##p<0.01.

FIG. 3: Schematic protocol for neutropenia mouse model

FIG. 4: CFU measured in blood, liver, kidney and spleen in a mouse model of febrile neutropenia, after administration with the listed compounds at the listed concentrations.

FIG. 5: Body weights measured in a mouse model of febrile neutropenia (with reference to Naïve control), after administration with the listed compounds at the listed concentrations.

FIG. 6: Amplification plot for samples from RT-PCT example.

FIG. 7: ¹H NMR spectrum of compound 1.

FIG. 8: LC/MS of compound 1.

FIG. 9: ¹H NMR spectrum of compound 2.1.

FIG. 10: LC/MS of compound 2.1.

FIG. 11: ¹H NMR spectrum of compound 2.2.

FIG. 12: LC/MS of compound 2.2.

FIG. 13: ¹H NMR spectrum of compound 2.3.

FIG. 14: LC/MS of compound 2.3.

### EXAMPLES

### Methods and Materials

MN8CampLuc cells were handled according to Nylén et. al. with the following exception when predifferention of cells were performed before induction:

Cell seeding was performed in medium where glucose was exchanged for galactose (5 mg/ml), which is known to promote differentiation in colon epithelial cells (Pinto, M., M. D. Appay, P. Simon-Assman, G. Chevalier, N. Dracopoli, J. Fogh, and A. Zweibaum, 1982, Biol. Cell., 44:193-196) Cells were then allowed to grow for 72 hours before stimulation with test compounds.

RT-PCR experiments were performed according to Nylen et. al. (Nylen F, Miraglia E, Cederlund A, Ottosson H, Stromberg R, Gudmundsson G H, Agerberth B. 2013. Boosting innate immunity: Development and validation of a cell-based screening assay to identify LL-37 inducers. Innate Immun.).

RT-PCR experiments for expression of marker genes for autophagy in HEK-293 cells were measured by real-time PCR. Data were normalized by the expression of the 18s rRNA housekeeping gene. For the immunofluorescence spectroscopy experiments HEK-293 cells were fixed after treatment with the inducers or control. The cells were then stained with DAPI to visualize the nuclei (blue), and immunolabeled with the anti-LC3, followed by the addition of Alexa-fluor 488 (green). Scale bar=10 μm.

All reagents and solvents (analytical grade) were purchased from commercial resources and were used without further purification. The NMR spectra were collected on a Bruker DRX-400 spectrometer (400 MHz for ¹H and 101 MHz for ¹³C) with the residual solvent signal as chemical shift reference. Mass spectra were recorded on a Micromass LCT (ESI-TOF) mass spectrometer. Pyridin-3-ylmethyl (4-((2-aminophenyl)carbamoyl)benzyl)carbamate (5, Entinostat) and N1-hydroxy-N8-phenyloctanediamide (12, Vorinostat) was purchased from LC laboratories (Woburn, MA, USA), N-(4-Methoxybenzyl)-1,2-benzenediamine (16) from Fluorochem Ltd (Hadfield, UK) and Trichostatin A (19) from Sigma-Aldrich Sweden AB (Stockholm, Sweden).

### Example 1. Synthesis of Compounds Relating to the Invention

Exemplary syntheses are provided below. Compounds of the invention may also be synthesised by other methods known in the art.

Compound 1 may be synthesised as follows:

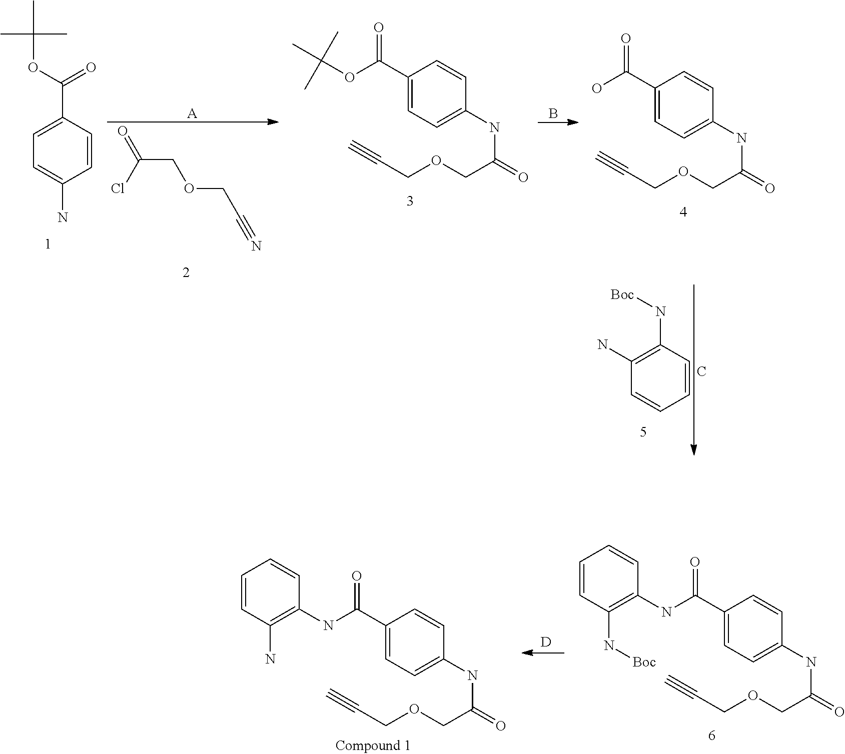

Step A: To a solution of 2 (1 equiv.) in acetonitrile was added CDI (1.2 equiv.). The reaction mixture was stirred for 40 min at r.t. and 1 (1 equiv.) was added. The resulting mixture was stirred overnight at 40° C., evaporated under reduced pressure, and diluted with ethyl acetate. The organic layer was washed with water, dried over anhydrous sodium sulfate and concentrated in vacuum. Purification of the residue via column chromatography on silica gel afforded 3.

Step B: To a solution of 3 in dichloromethane was added TFA (10 equiv.), and the reaction mixture was stirred overnight r.t. and evaporated under reduced pressure. The residue was crystallized from hexane to obtain 4.

Step C: To a solution of 4 in DMF was added triethylamine (2 equiv.) and HATU (1.1 equiv.), followed by 5 (1 equiv.). The reaction mixture was stirred overnight at r.t., diluted with water, and extracted ethyl acetate. The combined organic layers were washed with water, dried over anhydrous Na₂SO₄ and evaporated under reduced pressure to afford 6 which was used in the next step without further purification.

Step D: To a solution of 6 in dichloromethane was added TFA (10 equiv.), and the reaction mixture was stirred overnight r.t. and evaporated under reduced pressure. The residue was diluted with water and pH was adjusted to ˜8 with aq. solution of sodium bicarbonate. The precipitated product was isolated by simple filtration and washed with water and dried to obtain compound 1.

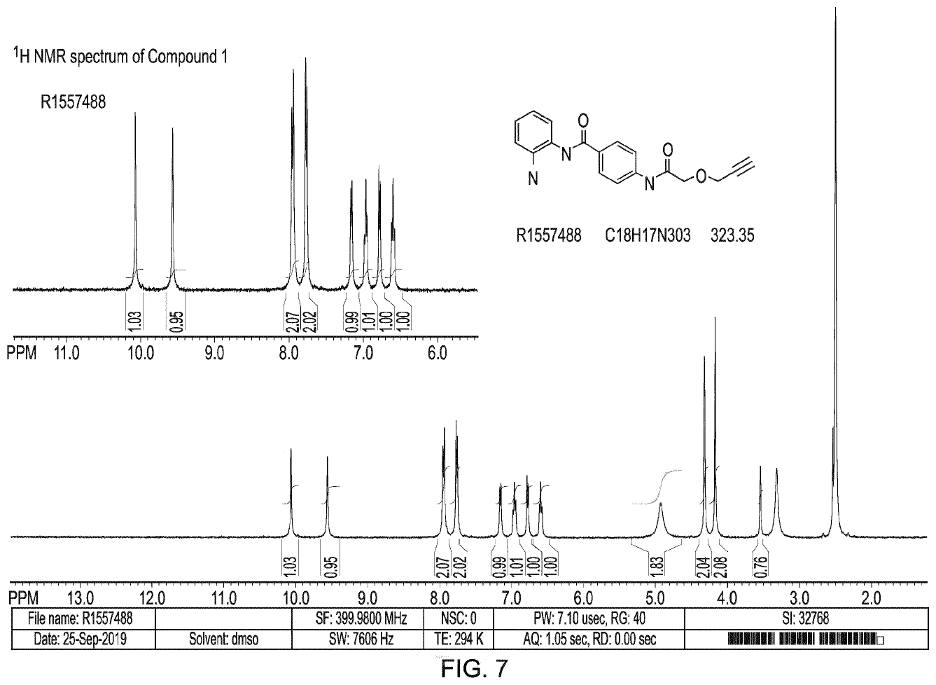

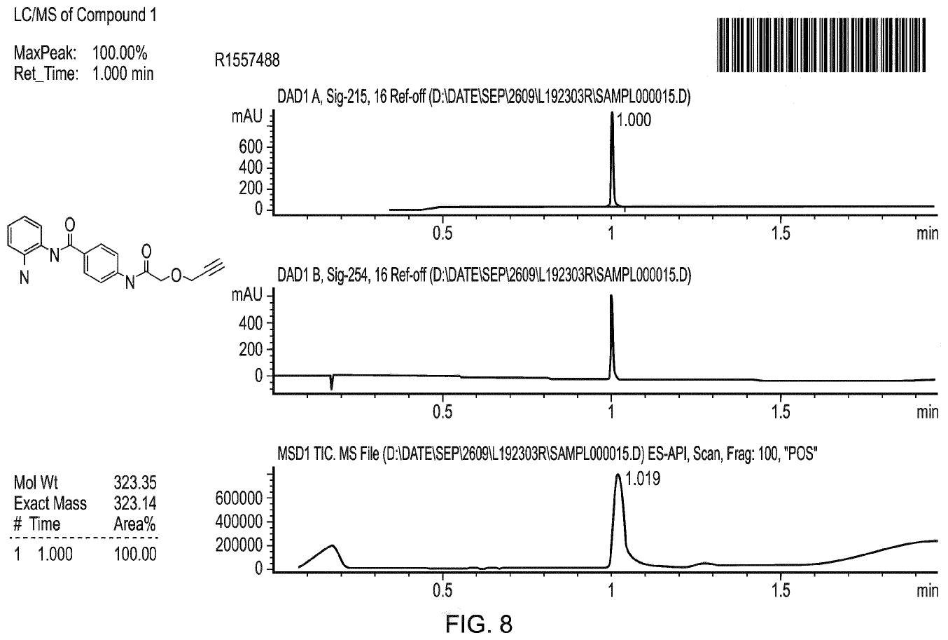

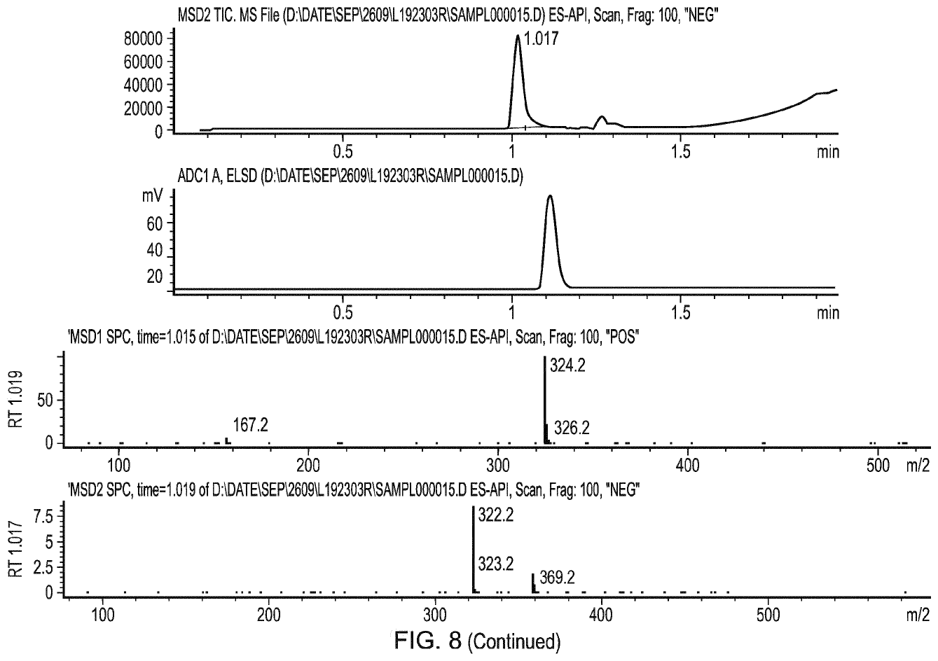

Analytical data for compound 1 is shown in FIGS. 7 and 8.

Compound 2.1 may be synthesised as follows:

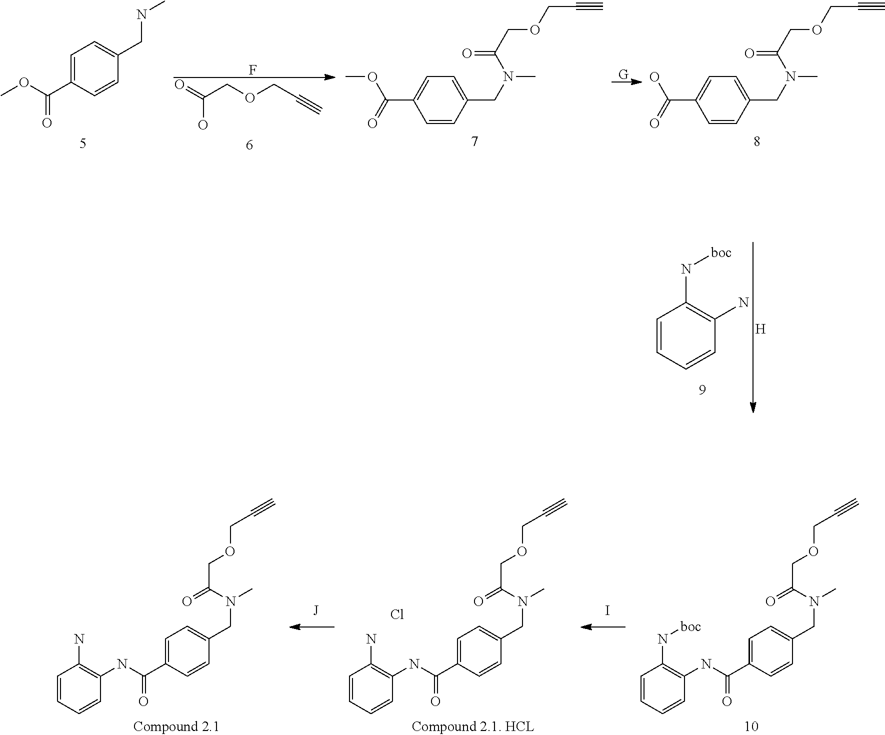

Step F: To the solution of 6 (3 g, 26.3 mmol) in dry DMF (50 mL) were added HATU (10.9 g, 28.6 mmol) and DIPEA (11.1 g, 85.8 mmol) and the mixture was stirred at r.t. for 30 min. Then 5 (6.16 g, 28.6 mmol, HCl salt) was added and the reaction mixture was stirred overnight at r.t. The solvent was evaporated, the residue was dissolved in EtOAc (100 mL) and washed with water (100 mL), aq. citric acid (10%, 100 mL) and aq. NaHCO₃ (100 mL). The organic layer was dried over Na₂SO₄ and evaporated under reduced pressure to obtain crude 7. After column chromatography (H:EA=1:1) pure ester 7 was obtain (5.55 g, 76%).

Step G: To a solution of 7 (5.55 g, 20.2 mmol) in MeOH (100 mL) was added NaOH (1.2 g, 30 mmol) in water (5 mL) and the reaction mixture was stirred overnight at r.t. The solvent was evaporated, the residue was diluted with water, acidified by citric acid, and extracted with EtOAc (3×100 mL). The combined organic layers were dried over Na₂SO₄ and evaporated under reduced pressure to obtain pure acid 8 (4.65 g, 88%).

Step H: To a solution of 8 (4.65 g, 17.8 mmol) in dry THF (50 mL) were added HATU (7.45 g, 19.6 mmol) and DIPEA (7.6 g, 58.7 mmol) and the mixture was stirred at r.t. for 30 min. Then 9 (4.1 g, 19.6 mmol) was added and the reaction mixture was stirred overnight at r.t. The solvent was evaporated, the residue was dissolved in EtOAc (100 mL) and washed with water (100 mL), aq. citric acid (10%, 100 mL) and aq. NaHCO₃ (100 mL). The organic layer was dried over Na₂SO₄ and evaporated under reduced pressure to obtain crude 10. After column chromatography (H:EA=1:4) pure ester 10 was obtain (5.6 g, 70%).

Step I: To a solution of 10 (5.6 g, 12.4 mmol) in dry dioxane (60 mL) was added HCl\Dioxane (60 mL) and the reaction mixture was stirred overnight at r.t. Then the resulting mixture was diluted with MTBE (100 mL), the solid was filtered and dried under reduced pressure to obtain compound 2.1 as HCl salt.

Step J: Compound 2.1 HCl salt (2 g) was dissolved in aq. NaHCO₃ (50 mL) and extracted with DCM (3×30 mL). Organic layers were dried over Na₂SO₄ and evaporated under reduced pressure to obtain compound 2.1.

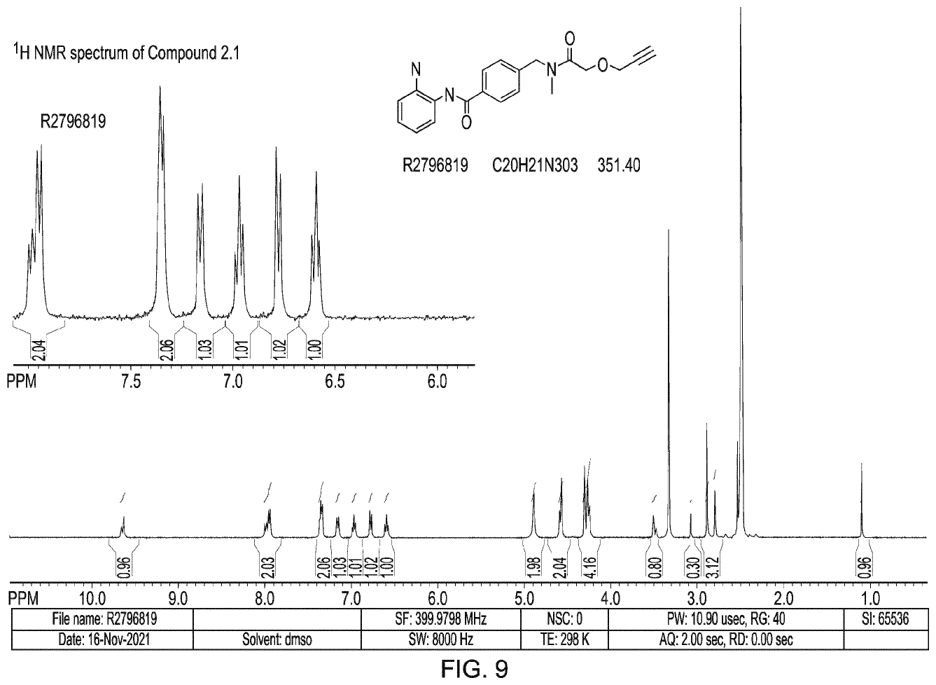

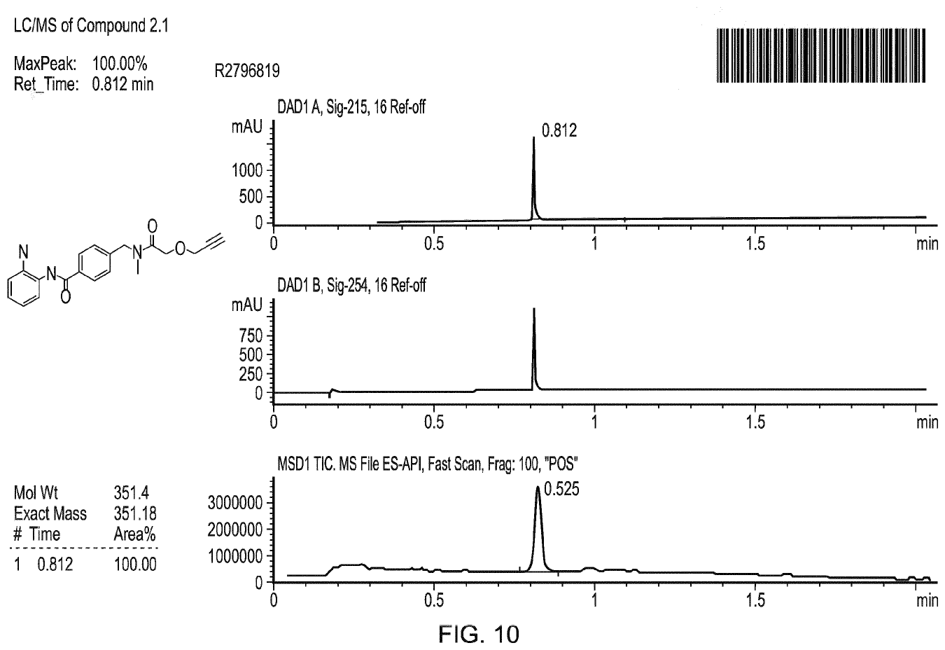

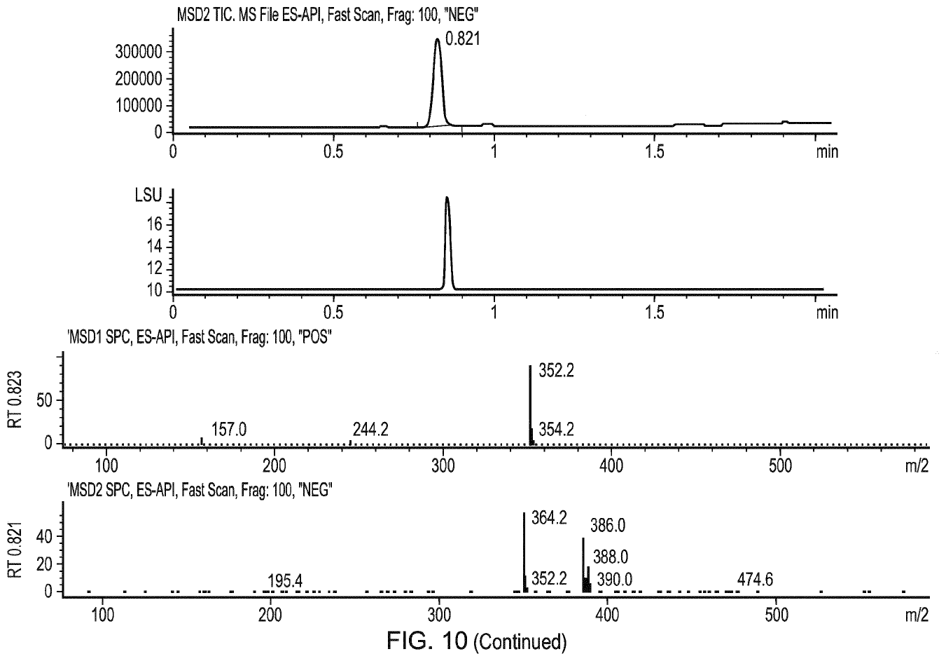

Analytical data for compound 2.1 is shown in FIGS. 9 and 10.

Compound 2.2 may be synthesised as follows:

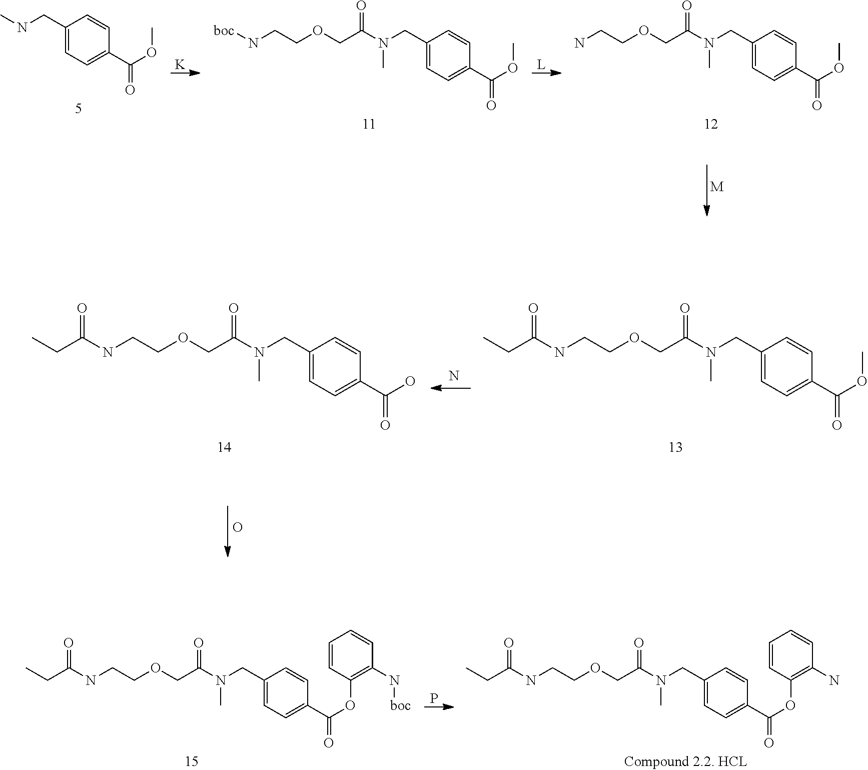

Step K: To a solution of 2-(2-((tert-butoxycarbonyl)amino)ethoxy)acetic acid (3 g, 13.7 mmol) in dry DMF (50 mL) were added HATU (5.7 g, 15 mmol) and DIPEA (5.8 g, 45.2 mmol) and the mixture was stirred at r.t. for 30 min. Then 5 (3.23 g, 15 mmol, HCl salt) was added and the reaction mixture was stirred overnight at r.t. The solvent was evaporated, the residue was dissolved in EtOAc (100 mL) and washed with water (100 mL), aq. citric acid (10%, 100 mL) and aq. NaHCO₃ (100 mL). The organic layer was dried over Na₂SO₄ and evaporated under reduced pressure to obtain crude 11. After column chromatography (H:EA=1:1) pure ester 11 was obtain (3 g, 57.5%).

Step L: To a solution of 11 (3 g, 7.89 mmol) in dry dioxane (30 mL) was added HCl\Dioxane (30 mL) and the reaction mixture was stirred overnight at r.t. The resulting mixture was diluted with MTBE (100 mL), the solid was filtered and dried under reduced pressure to obtain 12 as HCl salt.

Step M: To a suspension of 12 (1.8 g, 5.7 mmol) in dry THF (30 mL) was added TEA (2.3 g, 22.8 mmol) and the mixture was cooled to 0° C. Then propionyl chloride (0.63 g, 6.8 mmol) was added dropwise and the reaction mixture was stirred overnight at r.t. The solvent was evaporated, the residue was dissolved in DCM (50 mL) and washed with citric acid. The organic layer was dried over Na₂SO₄ and evaporated under reduced pressure to obtain pure 13 (1.9 g, 90%).

Step N: To a solution of 13 (1.9 g, 5.7 mmol) in MeOH (30 mL) was added NaOH (0.34 g, 8.5 mmol) in water (1 mL) and the reaction mixture was stirred overnight at r.t. The solvent was evaporated, the residue was diluted with water, acidified with citric acid, and extracted with EtOAc (3×100 mL). The combined organic layers were dried over Na₂SO₄ and evaporated under reduced pressure to obtain pure acid 14 (0.4 g, 25%).

Step O: To a solution of 14 (0.4 g, 1.2 mmol) in dry THF (10 mL) were added HATU (0.5 g, 1.3 mmol) and DIPEA (0.52 g, 4 mmol) and the mixture was stirred at r.t. for 30 min. Then 9 (see 9 in synthesis for compound 2.1) (0.27 g, 1.3 mmol) was added and the reaction mixture was stirred overnight at r.t. The solvent was evaporated, the residue was dissolved in EtOAc (100 mL) and washed with water (100 mL), aq. citric acid (10%, 100 mL) and aq. NaHCO₃ (100 mL). The organic layer was dried over Na₂SO₄ and evaporated under reduced pressure to obtain pure 15 was obtain (0.5 g, 75%).

Step P: To a solution of 15 (0.5 g, 0.98 mmol) in dry dioxane (10 mL) was added HCl\Dioxane (10 mL) and the reaction mixture was stirred overnight at r.t. Then the resulting mixture was diluted with MTBE (100 mL), the solid was filtered and dried under reduced pressure to obtain compound 2.2 as HCl salt.

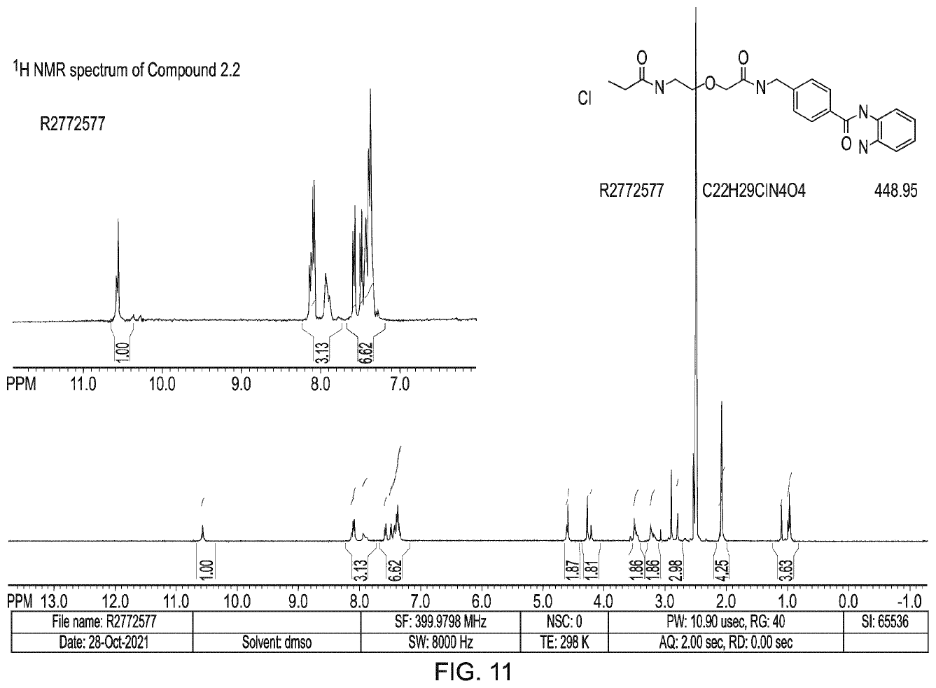

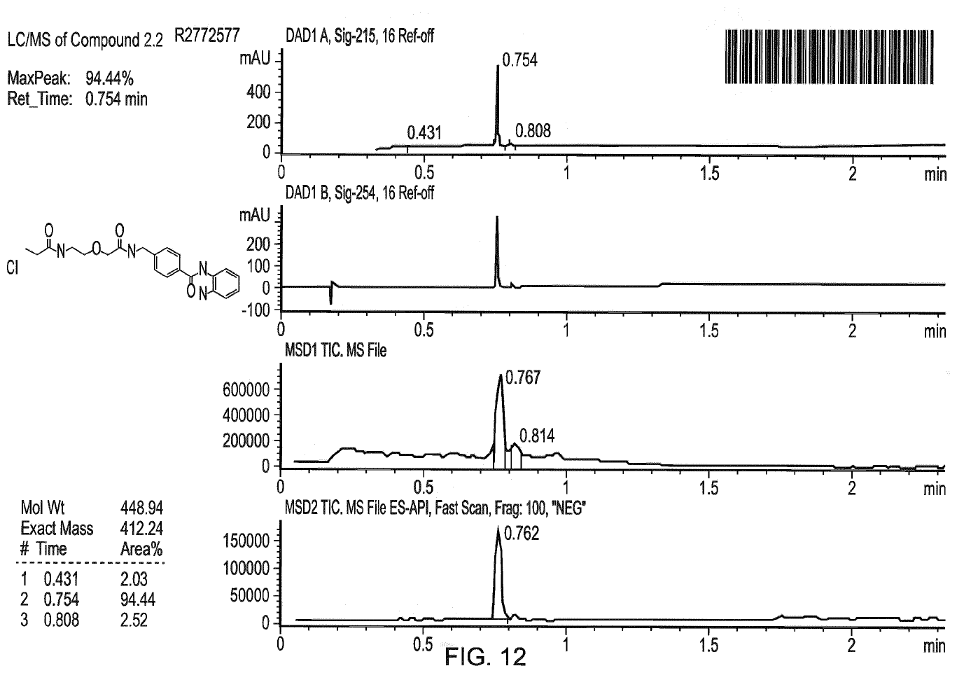

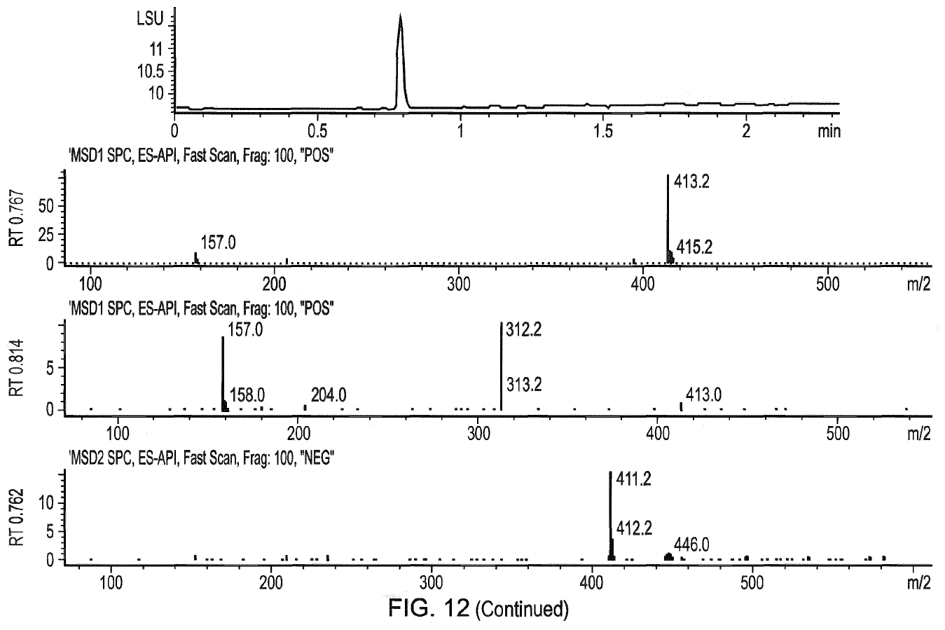

Analytical data for compound 2.2 is shown in FIGS. 11 and 12.

Compound 2.3 may be synthesised as follows:

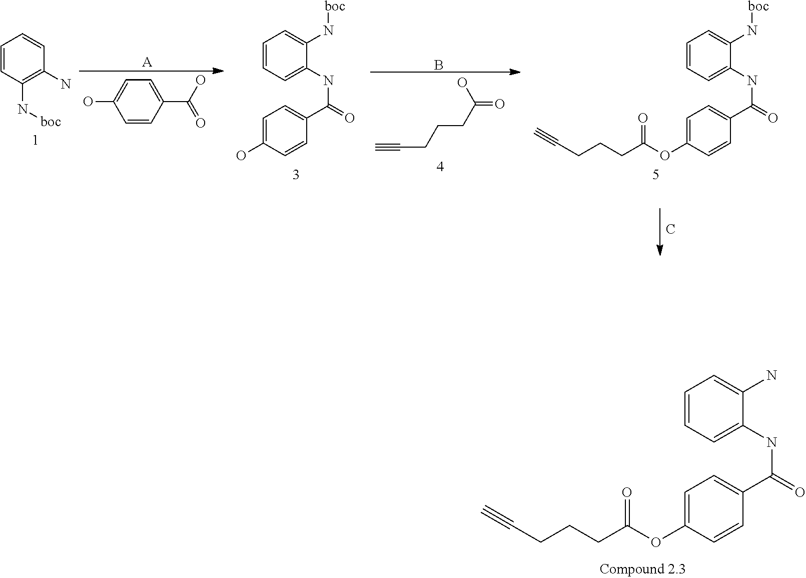

Step A: To a solution of 2 (3 g, 21.7 mmol) in dry THF (50 mL) were added HATU (9.1 g, 23.9 mmol) and DIPEA (9.3 g, 71.6 mmol) and the mixture was stirred at r.t. for 30 min. Then tert-butyl (2-aminophenyl)carbamate (1) (5 g, 23.9 mmol) was added and the reaction mixture was stirred overnight at r.t. After that, the solvent was evaporated, the residue was dissolved in EtOAc (100 mL) and washed with water (100 mL), ag. citric acid (10%, 100 mL) and aq. NaHCO₃ (100 mL). The organic layer was dried over Na₂SO₄ and evaporated under reduced (2.65 g) pressure to obtain crude 3. After column chromatography (H:EA=1:1) pure 3 was obtain (2.65 g).

Step B: To a solution of 3 (2.65 g, 8 mmol), 4 (1.32 g, 12 mmol) and DMAP (0.72 g, 5.9 mmol) in dry DMF (30 mL) was added DCC (2.43 g, 12 mmol) and the reaction mixture was stirred overnight at room temperature. Then the resulting mixture was quenched with water and extracted with EtOAc (3×50 mL). Organic layers were combined, washed with aq. NaHCO₃ and brine. Then dried over Na₂SO₄ and evaporated under reduced pressure. The crude was purified by column chromatography (CHCl₃:MeOH=24:1) to obtain pure 5 (1.5 g).

Step C: To a solution of 5 (1.5 g, 3.55 mmol) in dry dioxane (10 mL) was added HCl\Dioxane (20 mL) and the reaction mixture was stirred overnight at r.t. Then the resulting mixture was diluted with MTBE (50 mL), the solid was filtered and dried under reduced pressure to obtain compound 2.3 as HCl salt. The solid was dissolved in aq. NaHCO₃ and extracted with DCM (3×30 mL). Organic layers were dried over Na₂SO₄ and evaporated under reduced pressure to obtain compound 2.3.

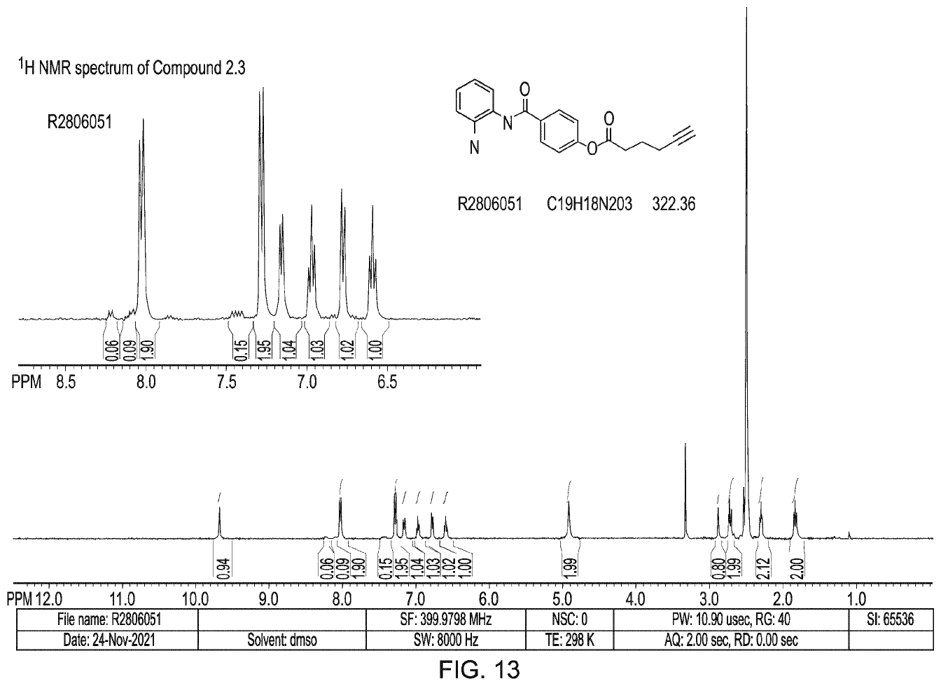

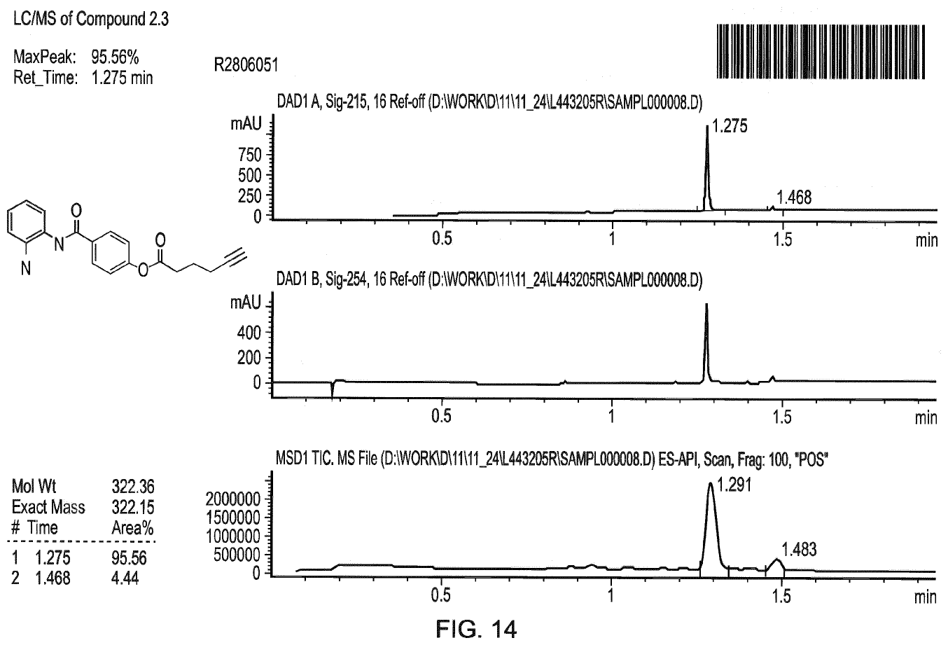

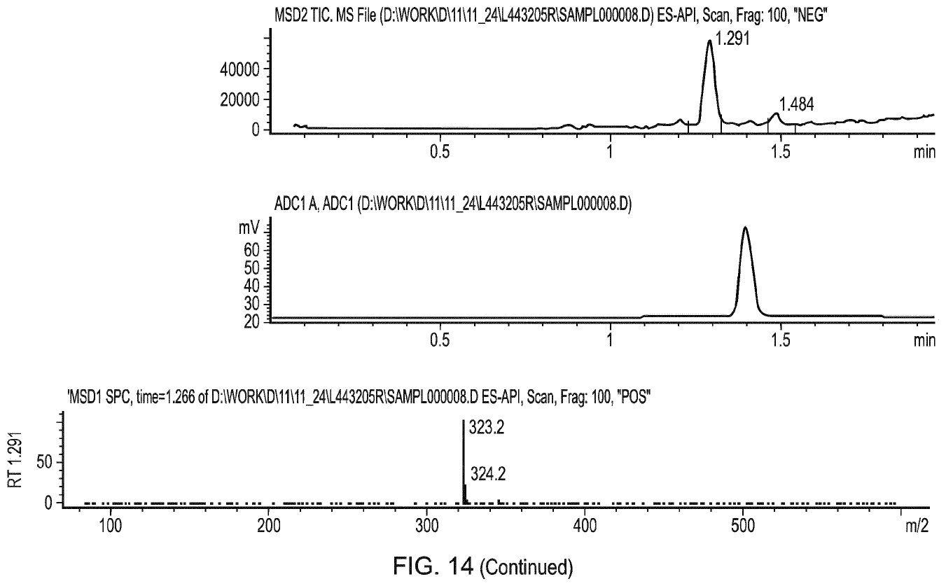

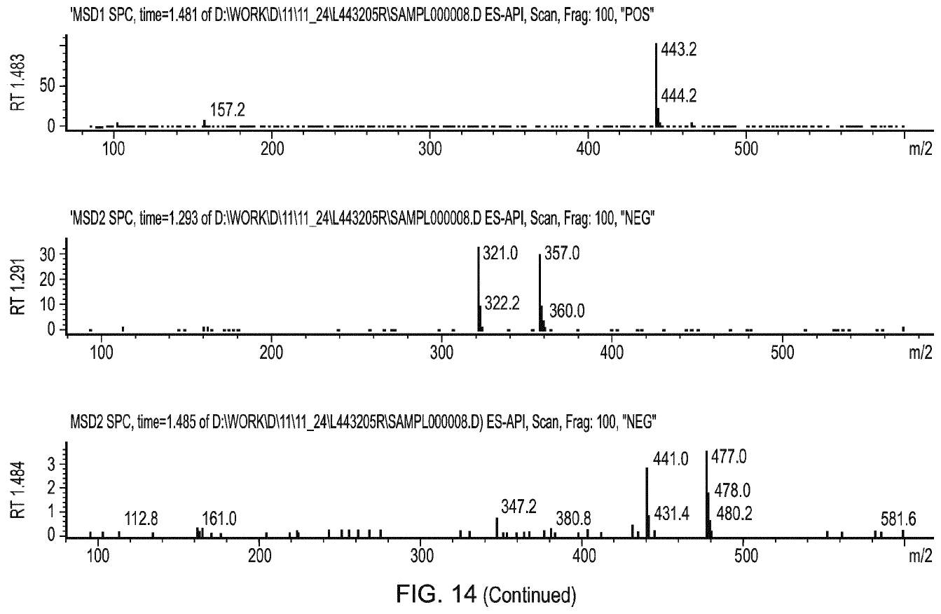

Analytical data for compound 2.3 is shown in FIGS. 13 and 14.

### Example 2. E. coli Mouse Infection Model

Compound 1 was administered orally to a mouse model of E. coli infection at 25 mg/kg, 73.5 mk/kg and 50 mg/kg. Compound 1 achieved the study goal of lowering Colony Unit Forming count in colon tissue by a factor of 3 log₁₀ compared to vehicle treated animals.

Unexpectedly, it was found that 1) Colony Forming Unit counts in liver and spleen turned out to be higher than expected (on the order of 2-3 log₁₀), and 2) Compound 1 had the unexpected effect of completely knocking out translocation of bacteria to these organs.

### Example 3: Evaluation of Further Compounds of the Invention

Further compounds were newly designed (compounds 2.1, 2.2 and 2.3), and were synthesised variants of compound 1.

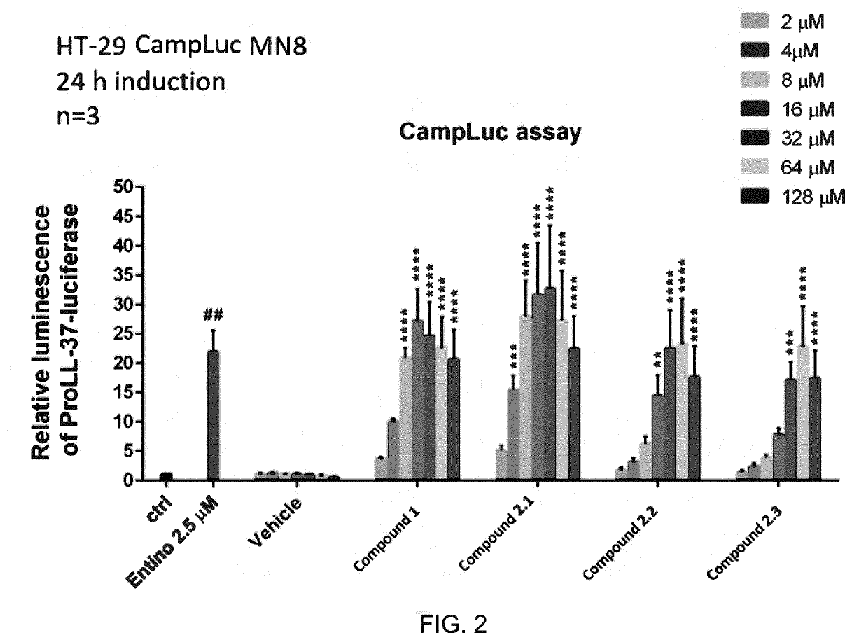

Compounds 2.1, 2.2 and 2.3 were tested for induction of antimicrobial peptides in a human cell line. The experiment yielded the dose dependent fold induction for expression of CAMP gene (encoding human cathelicidin), as shown in FIG. 2.

### Example 4: Comparison of Compound 1 to HO13 and HO56

Compound 1 was selected as a particularly preferred embodiment of the invention, after comparison with HO53 and HO56 (Myszor, I. T., Parveen, Z., Ottosson, H. et al. Novel aroylated phenylenediamine compounds enhance antimicrobial defense and maintain airway epithelial barrier integrity. Sci Rep 9, 7114 (2019). https://doi.org/10.1038/s41598-019-43350-z):

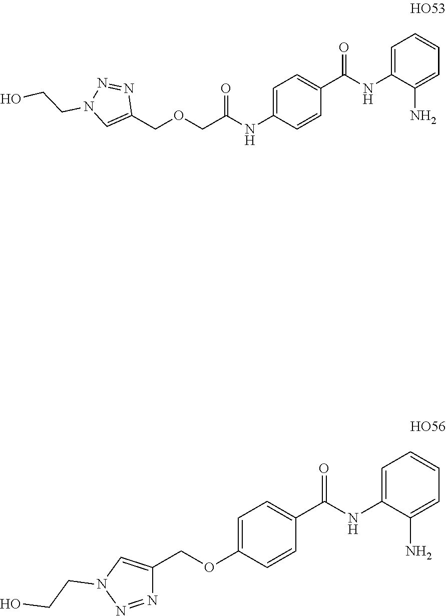 [SMILES: Nc1ccccc1NC(=O)c1ccc(NC(=O)COCc2cn(CCO)nn2)cc1 ; Nc1ccccc1NC(=O)c1ccc(OCc2cn(CCO)nn2)cc1]

Table 1 below depicts comparison data for the three compounds. An important characteristic of compound 1 vs HO53 and HO56 is that it unexpectedly displays high affinity (permeability) in a Caco-2 cell study. Caco-2 cells are used as a model of the intestinal epithelial barrier. The results thus show that the compounds of the invention are particularly beneficial in treating diseases that can be treated by improving or restoring gastrointestinal barrier function, and/or preventing or reducing microbial translocation through the gastrointestinal barrier.

TABLE 1

| Compound | HO53 | | HO56 | | Compound 1 | |
|---|---|---|---|---|---|---|
| Caco-2 Permeability | Low | | Low | | High | |
| A-B dir. | | | | | | |
| Active efflux | Yes | | Yes | | No | |
| Plasma stability Mouse | Low | | High | | Low | |
| Plasma stability Rat | High | | High | | High | |
| Stability SGD, | High | | High | | High | |
| SIF w. Enzymes | | | | | | |
| Dose Esc. Tox | >100 | mg/kg | 10 | mg/kg | 100 | mg/kg |
| mouse iv | | | | | | |
| Dose Esc. Tox | >100 | mg/kg | >200 | mg/kg | 50 | mg/kg |
| mouse PO | | | | | | |
| 7 Days PO once daily | >50 | mg/kg | >50 | mg/kg | >50 | mg/kg |
| MTD - mouse | | | | | | |
| 7 Days PO twice daily | | | | | 50 | mg/kg |
| MTD - mouse | | | | | | |
| Bioavailability rat | 3% | | 16% | | 1% | |
| C_max IV - rat | 21 | μM | 26 | μM | 177 | μM |
| C_max PO - rat | 0.09 | μM | 1.2 | μM | 3.3 | μM |
| V-d IV - rat | 1700 | ml/kg | 3070 | ml/kg | 565 | ml/kg |
| Elimination T½ (min) | 35 | | 132 | | 34 | |
| PO - rat | | | | | | |
| Elimination T½ (min) | 126 | | 94 | | 66 | |
| PIV - rat | | | | | | |
| PO single 5 mg/kg | | | | | 0.9 | μM |
| mouse - Ileum | | | | | | |
| C-max | | | | | | |
| PO single 15 mg/kg | | | | | 2.0 | μM |
| mouse - Ileum | | | | | | |
| C-max | | | | | | |
| PO single 50 mg/kg | | | | | 7.1 | μM |
| mouse - Ileum | | | | | | |
| C-max | | | | | | |

### Example 5: Mouse Model Results

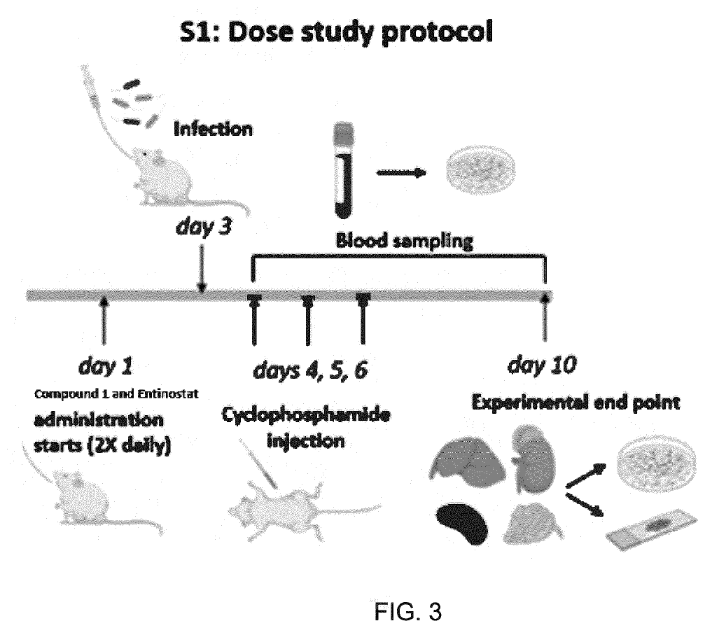

A mouse model was prepared to mimic chemically induced febrile neutropenia. A schematic of the experimental model is depicted in FIG. 3.

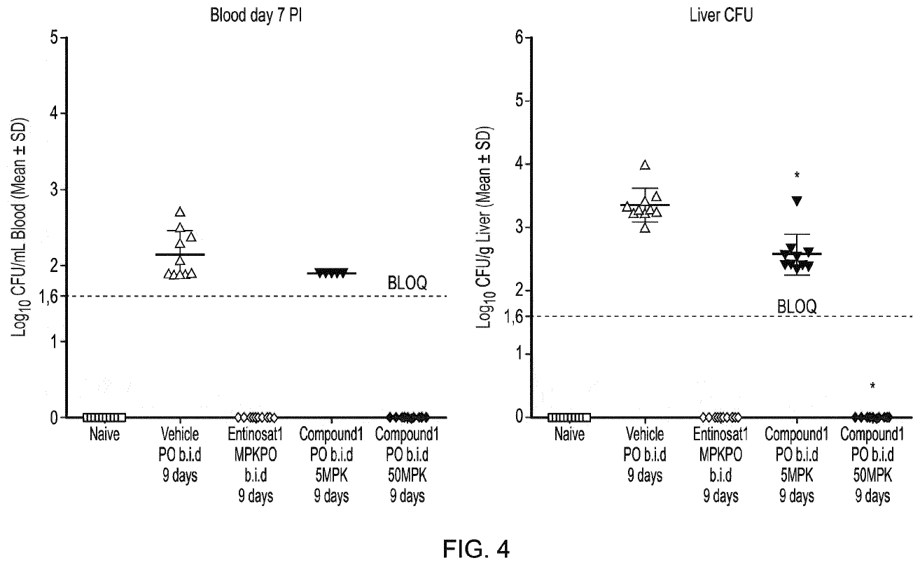

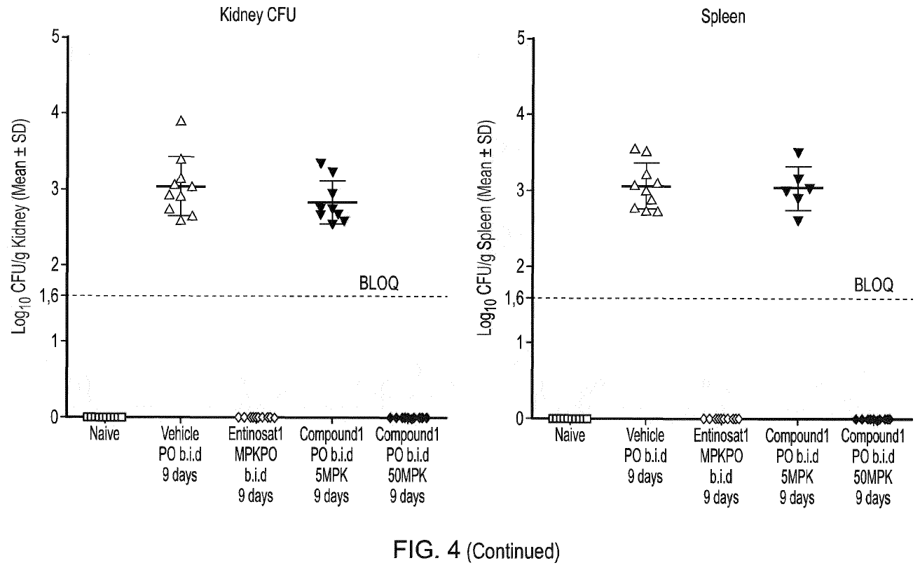

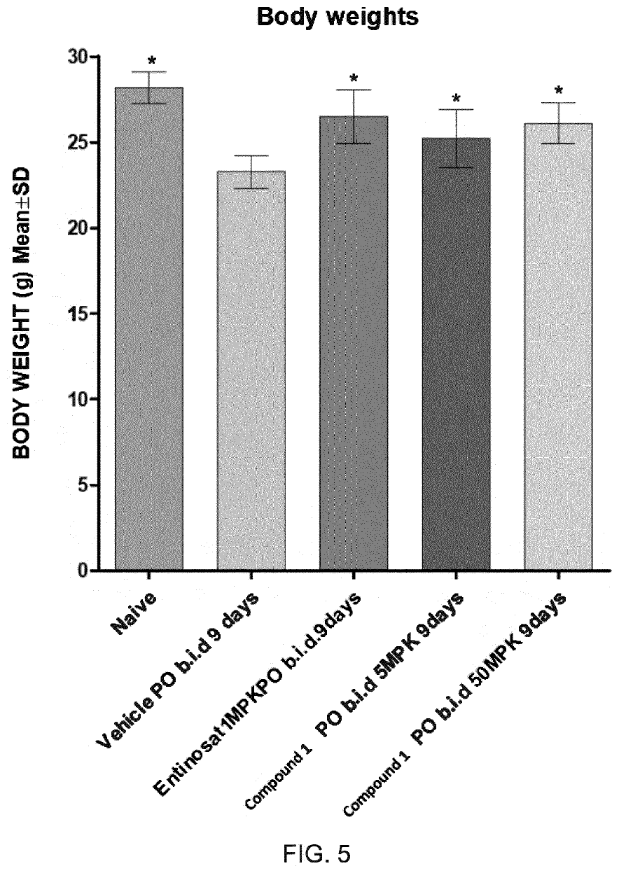

The results of this experiment are shown in Tables 2-7 and FIGS. 4 and 5. Certain observations include:
Animals were apparently normal in Naïve and compound 1 and Entinostat treated groups. Animals in vehicle control observed lethargic on day 7
Clinical signs were consistent with efficacy.
CFU Load in blood was BLOQ in all the groups on day 5 & 6
compound 1 showed significant dose dependent antibacterial effect in liver and blood when compared to their corresponding vehicle controls (p<0.05) on day 7
Bacteria was BLOQ in animals treated with Entinostat and compound 1 (50 mg/kg) on day 7
Colonies recovered from plates were confirmed to Enterotoxigenic Escherichia coli by RT-PCR.

TABLE 2
Efficacy of compound 1 against Intestinal Bacterial Translocation of Enterotoxigenic
Escherichia coli (ETEC) (ATCC ® 35401 ™, H10407) in a Neutropenic Mouse Model.

| | | | | | Body |
|---|---|---|---|---|---|
| | | | | | weights |
| | Log₁₀ CFU/g^{#}[Mean ± SD] | | | | (Final Day) |
| Group | Blood | Liver | Spleen | Kidneys | (Mean ± SD) |
| Naïve [none] | BLOQ | BLOQ | BLOQ | BLOQ | 28.2 ± 0.92* |
| Vehicle Control DMSO: | 2.15 ± 0.31 | 3.35 ± 0.26 | 3.07 ± 0.3 | 3.04 ± 0.39 | 23.3 ± 0.95 |
| PEG400: dH2O | | | | | |
| (20%:50%:30%, v/v) | | | | | |
| PO, twice | | | | | |
| daily(q12h), | | | | | |
| 7 days | | | | | |
| Entinostat (Positive | BLOQ | BLOQ | BLOQ | BLOQ | 26.5 ± 1.58* |
| control) | | | | | |
| 1 mg/kg, PO, | | | | | |
| twice daily(q12h), | | | | | |
| 9 days | | | | | |
| Compound 1 | 1.9 ± 0* | 2.58 ± 0.32* | 3.05 ± 0.3^{ns} | 2.83 ± 0.29^{ns} | 25.2 ± 1.69* |
| 5 mg/kg, | | | | | |
| PO, twice | | | | | |
| daily(q12h), | | | | | |
| 9 days | | | | | |
| Compound 1 | BLOQ | BLOQ | BLOQ | BLOQ | 26.1 ± 1.20* |
| 50 mg/kg, | | | | | |
| PO, twice | | | | | |
| daily(q12h), | | | | | |
| 9 days | | | | | |

TABLE 3
Clinical observations in a mouse model of febrile neutropenia
(with reference to Naïve control), after administration with the
listed compounds at the listed concentrations.
Clinical observation (Mean Score)

| | | | | Entinostat | Compound 1, | Compound 1, |
|---|---|---|---|---|---|---|
| | | | Vehicle, | [1 mg/kg, | 5 mg/kg, | 50 mg/kg, |
| | | | PO, twice | PO, twice | PO, twice | PO, twice |
| | Clinical | Naïve | daily(q12h), | daily(q12h), | daily(q12h), | daily(q12h), |
| Days | signs | [none] | 9 days | 9 days] | 9 days | 9 days |
| Day (−2) | Rough coat | 0.0 | 0.0 | 0.0 | 0.0 | 70.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (−1) | Rough coat | 0.0 | 0.0 | 0.0 | 0.0 | 0.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (0) | Rough coat | 0.0 | 0.0 | 0.0 | 0.0 | 0.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (1) | Rough coat | 0.0 | 0.0 | 0.0 | 0.0 | 0.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (2) | Rough coat | 0.0 | 0.0 | 0.0 | 0.0 | 0.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (3) | Rough coat | 0.0 | 0.0 | 0.0 | 0.0 | 0.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (4) | Rough coat | 0.0 | 0.0 | 0.0 | 0.0 | 0.0 |
| | Hunched | 0.0 | 0.0 | 0.0 | 0.0 | 0.0 |
| | Posture | | | | | |
| | Lethergy | 0.0 | 0.0 | 0.0 | 0.0 | 0.0 |
| | hyperpnea | 0.0 | 0.0 | 0.0 | 0.0 | 0.0 |
| Day (5) | Rough coat | 0.0 | 0.6 | 0.1 | 0.5 | 0.0 |
| | Hunched | 0.0 | 0.0 | 0.0 | 0.0 | 0.0 |
| | Posture | | | | | |
| | Lethergy | 0.0 | 0.3 | 0.0 | 0.1 | 0.0 |
| | hyperpnea | 0.0 | 0.0 | 0.0 | 0.0 | 0.0 |
| Day (6) | Rough coat | 0.0 | 1.0 | 0.1 | 0.7 | 0.4 |
| | Hunched | 0.0 | 0.0 | 0.0 | 0.0 | 0.0 |
| | Posture | | | | | |
| | Lethergy | 0.0 | 0.5 | 0.0 | 0.4 | 0.0 |
| | hyperpnea | 0.0 | 0.0 | 0.0 | 0.0 | 0.0 |
| Day (7) | Rough coat | 0.0 | 1.0 | 0.2 | 1.0 | 0.4 |
| | Hunched | 0.0 | 0.0 | 0.0 | 0.0 | 0.0 |
| | Posture | | | | | |
| | Lethergy | 0.0 | 0.7 | 0.0 | 0.5 | 0.0 |
| | hyperpnea | 0.0 | 0.0 | 0.0 | 0.0 | 0.0 |

0 = No or absent;
1 = Yes or Present

TABLE 4
Liver-Raw data on Vancomycin (32 μg/ml) plates, Log10 CFU/g

| | Vehicle Control DMSO: | Entinostat | Compound 1, | Compound 1, |
|---|---|---|---|---|
| | PEG400: dH₂O | 1 mg/kg, | 5 mg/kg, | 50 mg/kg, |
| | PO, twice | PO, twice | PO, twice | daily(q12h), |
| Naïve | daily(q12h), | daily(q12h), | daily(q12h), | PO, twice |
| (none) | 9 days | 9 days | 9 days | 9 days |
| BLOQ | 3.49 | BLOQ | 2.41 | BLOQ |
| BLOQ | 4.00 | BLOQ | 2.67 | BLOQ |
| BLOQ | 3.41 | BLOQ | 2.41 | BLOQ |
| BLOQ | 3.33 | BLOQ | 3.42 | BLOQ |
| BLOQ | 3.25 | BLOQ | 2.33 | BLOQ |
| BLOQ | 3.01 | BLOQ | 2.39 | BLOQ |
| BLOQ | 3.27 | BLOQ | 2.39 | BLOQ |
| BLOQ | 3.24 | BLOQ | 2.53 | BLOQ |
| BLOQ | 3.29 | BLOQ | 2.61 | BLOQ |
| BLOQ | 3.22 | BLOQ | 2.57 | BLOQ |

TABLE 5
Spleen -Raw data on Vancomycin (32 μg/ml) plates, Log10 CFU/g

| | Vehicle Control DMSO: | Entinostat | Compound 1, | Compound 1, |
|---|---|---|---|---|
| | PEG400: dH₂O | 1 mg/kg, | 5 mg/kg, | 50 mg/kg, |
| | PO, twice | PO, twice | PO, twice | PO, twice |
| Naïve | daily(q12h), | daily(q12h), | daily(q12h), | daily(q12h), |
| (none) | 9 days | 9 days | 9 days | 9 days |
| BLOQ | 2.75 | BLOQ | 3.17 | BLOQ |
| BLOQ | 2.89 | BLOQ | 3.04 | BLOQ |
| BLOQ | 3.11 | BLOQ | 3.00 | BLOQ |
| BLOQ | 2.78 | BLOQ | 3.51 | BLOQ |
| BLOQ | 3.56 | BLOQ | 2.62 | BLOQ |
| BLOQ | 3.02 | BLOQ | BLOQ | BLOQ |
| BLOQ | 3.52 | BLOQ | 2.91 | BLOQ |
| BLOQ | 2.75 | BLOQ | BLOQ | BLOQ |
| BLOQ | 3.08 | BLOQ | BLOQ | BLOQ |
| BLOQ | 3.23 | BLOQ | BLOQ | BLOQ |

TABLE 6
Kidney -Raw data on Vancomycin (32 μg/ml) plates, Log10 CFU/g

| | Vehicle Control DMSO: | Entinostat | Compound 1, | Compound 1, |
|---|---|---|---|---|
| | PEG400: dH₂O | 1 mg/kg, | 5 mg/kg, | 50 mg/kg, |
| | PO, twice | PO, twice | PO, twice | PO, twice |
| Naïve | daily(q12h), | daily(q12h), | daily(q12h), | daily(q12h), |
| (none) | 9 days | 9 days | 9 days | 9 days |
| BLOQ | 3.40 | BLOQ | BLOQ | BLOQ |
| BLOQ | 3.15 | BLOQ | 2.73 | BLOQ |
| BLOQ | 3.89 | BLOQ | 3.23 | BLOQ |
| BLOQ | 2.91 | BLOQ | 2.66 | BLOQ |
| BLOQ | 2.59 | BLOQ | 2.94 | BLOQ |
| BLOQ | 2.92 | BLOQ | 3.34 | BLOQ |
| BLOQ | 3.06 | BLOQ | 2.68 | BLOQ |
| BLOQ | 2.73 | BLOQ | 2.59 | BLOQ |
| BLOQ | 3.04 | BLOQ | 2.54 | BLOQ |
| BLOQ | 2.65 | BLOQ | 2.75 | BLOQ |

TABLE 7
Blood -Raw data on Vancomycin (32 μg/ml) plates, Log10 CFU/g - on day 7 PI

| | Vehicle Control DMSO: | Entinostat | Compound 1, | Compound 1, |
|---|---|---|---|---|
| | PEG400: dH₂O | 1 mg/kg, | 5 mg/kg, | 50 mg/kg, |
| | PO, twice | PO, twice | PO, twice | PO, twice |
| Naïve | daily(q12h), | daily(q12h), | daily(q12h), | daily(q12h), |
| (none) | 9 days | 9 days | 9 days | 9 days |
| BLOQ | 2.30 | BLOQ | 1.90 | BLOQ |
| BLOQ | 2.51 | BLOQ | 1.90 | BLOQ |
| BLOQ | 1.90 | BLOQ | BLOQ | BLOQ |
| BLOQ | 2.72 | BLOQ | BLOQ | BLOQ |
| BLOQ | 1.90 | BLOQ | 1.90 | BLOQ |
| BLOQ | 1.90 | BLOQ | 1.90 | BLOQ |
| BLOQ | 1.90 | BLOQ | BLOQ | BLOQ |
| BLOQ | 2.38 | BLOQ | BLOQ | BLOQ |
| BLOQ | 2.08 | BLOQ | 1.90 | BLOQ |
| BLOQ | 1.90 | BLOQ | BLOQ | BLOQ |

### Example 6: RT-PCR Data

RNA Extraction: RNA from representative bacterial colonies on plates were extracted by Nucleo-Pore® RNASure® Mini Kit. Final volume of RNA eluted was 30 μl. Further these sample RNAs were processed for qRT-PCR.

Quantitative real-time polymerase chain reaction (qRT-PCR): Equal volume of RNA obtained was used in setting up the qRT-PCR reaction and RNA-Direct™ SYBR® Green Realtime PCR Master Mix kit was used. Further samples were analysed on a QuantStudio™ 3 Real-Time PCR System.

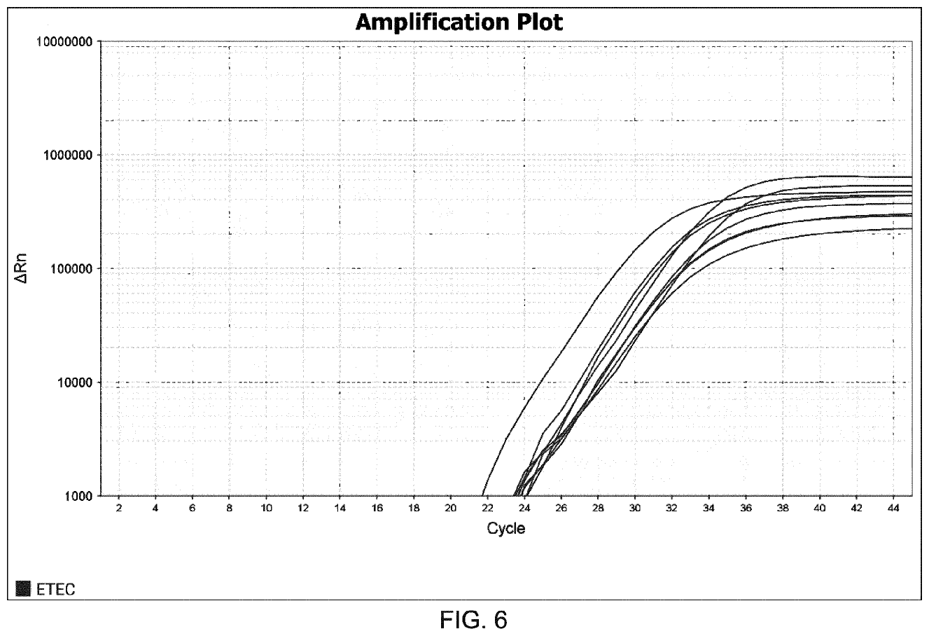

Results: As shown in Table 8 and FIG. 6, RT-PCR data, confirms colonies recovered from plates are Enterotoxigenic Escherichia coli.

TABLE 8

| CFU/ml- | |
|---|---|
| Samples | Ct Values |
| #13 spleen | 29.17 |
| #34 spleen | 30.24 |
| #13 kidney | 28.48 |
| #14 kidney | 29.69 |
| #33 kidney | 28.74 |
| #36 kidney | 29.69 |
| #12 Liver | 30.03 |
| #34 Liver | 29.69 |
| # 14 Blood | 26.58 |

### Example 7: Summary of Experiments

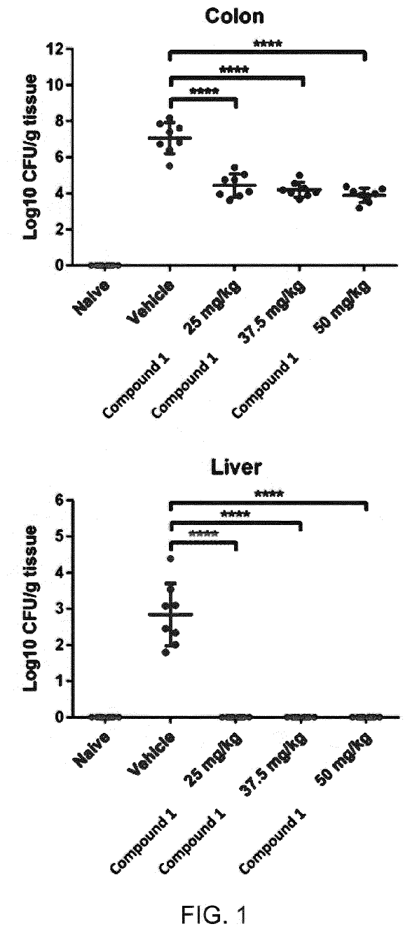

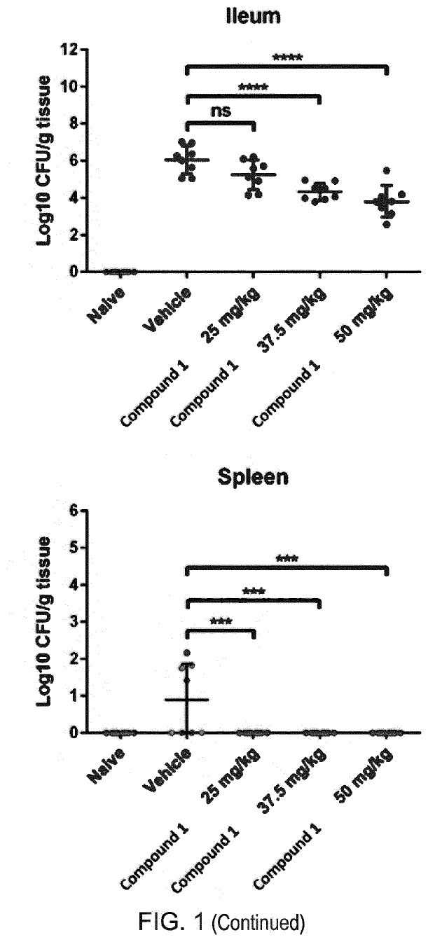

Infecting mice through oral administration of a vancomycin resistant pathogenic strain of E. coli simplified the process of determining the mechanism(s) by which compound 1 exerts its effects on the pathogenic process. All bacteria grown and detected in these experiments are not commensal bacteria because they are killed and do not grow on bacteria plates that have vancomycin.
Compound 1 is not acting as an antibiotic nor is it stimulating the production of innate antimicrobial agents to such an extent that they function as general antibiotics. This is evidenced by the fact that there still are viable vancomycin resistant E. coli present in the colon and ileum at the conclusion of the experiment (FIG. 1). The E. coli burden in the intestines is reduced by close to 3-logs compared to control, which indicates that the growth of the E. coli is attenuated, most likely through stimulation of innate antimicrobial peptide production. This is supported by in vitro data which demonstrates that compound 1 is not toxic to bacteria in culture and that innate antimicrobial peptides function like general antibiotics only at super physiological concentrations.
FIG. 1 also demonstrates that the inhibition of vancomycin resistant E. coli translocation to the liver and spleen is not compound 1 dose dependent. In contrast, compound 1 shows dose dependence in the attenuation of E. coli growth in the colon and ileum. It is therefore unlikely that compound 1 is inhibiting E. coli translocation to these organs solely due to its ability to stimulate production of antibacterial peptides. The most likely explanation is the ability of compound 1 and related compounds to stimulate and strengthen the cell-cell contacts, as has been demonstrated by in vitro cell culture experiments. This is supported by the data presented in FIG. 4. That experiment was performed in the presence of cyclophosphamide, which is known to cause febrile neutropenia due to translocation of bacteria to vital organs. In FIG. 4, the lower concentration of compound 1 (5 mg/kg) is having little to no effect on bacterial translocation while the higher concentration (50 mg/kg) inhibits it completely.
Taken together these data suggest two cooperating but separate mechanisms are responsible for the actions of compound 1 on E. coli pathogenicity in the intestines. First is the ability of the compound 1 to attenuate the growth of bacteria and secondly is its ability to inhibit the bacterial translocation.

### Example 8: HDAC Inhibition Data

The purpose of the study is to determine the effects of Compound 1 on the enzymatic activities of recombinant human HDAC1, HDAC2, HDAC3, HDAC4, HDAC5, HDAC6, HDAC7, HDAC8, and HDAC9 using in-vitro enzymatic assays.

Materials were as follows:
HDAC Assay Buffer (BPS catalog number 50031)
HDAC Assay Developer (BPS catalog number 50030)
HDAC Substrate 3 (BPS number 50037)
HDAC Class 2a Substrate 1 (BPS number 50040)
TSA was purchased from Selleck (Houston, TX, Catalog number S1045)
SAHA was purchased from Cayman Chemicals (Ann Arbor, MI, Catalog Number 10009929)

Compounds were as follows:

| Compound | Compound | Stock | Dissolving | Test Range | Intermediate |
|---|---|---|---|---|---|
| I.D. | Supplied | Concentration | Solvent | (μM) | Dilution |
| AKT-011 | Solid | 20 mM | DMSO | 1-200 | 10% DMSO in |
| | | | | | Assay Buffer |
| SAHA* | Solution | 10 mM | DMSO | 0.003-100 | 10% DMSO in |
| | | | | | Assay Buffer |
| TSA* | Solution | 10 mM | DMSO | 0.003-100 | 10% DMSO in |
| | | | | | Assay Buffer |

*Reference Compounds

Experimental conditions were as follows:

| | | | Enzyme Used | |
|---|---|---|---|---|
| Assay | Catalog# | Enzyme Lot# | (ng/rxn) | Substrate |
| HDAC1 | 50051 | 200917-1 | 8 | 20 uM HDAC Substrate 3 |
| HDAC2 | 50002 | 150701 | 12 | 20 uM HDAC Substrate 3 |
| HDAC3 | 50003 | 190326 | 5 | 20 uM HDAC Substrate 3 |
| HDAC4 | 50004 | 130828-G | 1 | 2 uM HDAC Substrate 2A |
| HDAC5 | 50005 | 201217-G3 | 30 | 2 uM HDAC Substrate 2A |
| HDAC6 | 50006 | 220419-GC | 20 | 20 uM HDAC Substrate 3 |
| HDAC7 | 50007 | 150825A | 2 | 2 uM HDAC Substrate 2A |
| HDAC8 | 50008 | 161220 | 20 | 2 uM HDAC Substrate 2A |
| HDAC9 | 50009 | 150520-G3 | 5 | 2 uM HDAC Substrate 2A |

Assay conditions were as follows:

All of the compounds were dissolved in DMSO. A series of dilutions of the compounds were prepared with 10% DMSO in HDAC assay buffer and 5 μl of the dilution was added to a 50 μl reaction so that the final concentration of DMSO was 1% in all of reactions. The compounds were pre-incubated in duplicate at RT for 30 minutes in a mixture containing HDAC assay buffer, 5 μg BSA, HDAC enzyme and a test compound. After 30 minutes, the enzymatic reactions were initiated by the addition of HDAC substrate to a final concentration of 20 μM or 2 μM. The enzymatic reaction proceeded for 30 minutes at 37° C. After enzymatic reactions, 50 μl of 2×HDAC Developer was added to each well for the HDAC enzymes and the plate was incubated at room temperature for an additional 15 minutes. Fluorescence intensity was measured at an excitation of 360 nm and an emission of 460 nm using a Tecan Infinite M1000 microplate reader.

Data analysis was as follows:

HDAC activity assays were performed in duplicates at each concentration. The fluorescent intensity data were analyzed using the computer software, Graphpad Prism. In the absence of the compound, the fluorescent intensity (Ft) in each data set was defined as 100% activity. In the absence of HDAC, the fluorescent intensity (Fb) in each data set was defined as 0% activity. The percent activity in the presence of each compound was calculated according to the following equation: % activity=(F−Fb)/(Ft−Fb), where F=the fluorescent intensity in the presence of the compound. The values of % activity versus a series of compound concentrations were then plotted using non-linear regression analysis of Sigmoidal dose-response curve generated with the equation Y=B+(T−B)/1+10\hat{ }((Log EC50−X)×Hill Slope), where Y=percent activity, B=minimum percent activity, T=maximum percent activity, X=logarithm of compound and Hill Slope=slope factor or Hill coefficient. The IC50 value was determined by the concentration causing a half-maximal percent activity.

A summary of the effects of the compounds on HDAC activities is as follows:

IC₅₀ (μM)

| Enzymes | AKT-011 | SAHA | TSA |
|---|---|---|---|
| HDAC1 | IC₅₀ < 1 μM | 0.087 | — |
| | 75% Inhib. @ 1 μM | | |
| HDAC2 | ~ 1.2 | 0.39 | — |
| HDAC3 | IC₅₀ ≤ 1 μM | 0.087 | — |
| | 61% Inhib. @ 1 μM | | |
| HDAC4 | IC₅₀ > 200 μM | — | 6.7 |
| | No Inhib. @ 200 μM | | |
| HDAC5 | IC₅₀ > 200 μM | — | 4.6 |
| | No Inhib. @ 200 μM | | |
| HDAC6 | IC₅₀ > 200 μM | 0.041 | — |
| | No Inhib. @ 200 μM | | |
| HDAC7 | IC₅₀ > 200 μM | — | 3.6 |
| | No Inhib. @ 200 μM | | |
| HDAC8 | IC₅₀ > 200 μM | — | 1.8 |
| | 42% Inhib. @ 200 μM | | |
| HDAC9 | IC₅₀ > 200 μM | — | 4.0 |
| | No Inhib. @ 200 μM | | |

### REFERENCES

1. Original Articles Epidemiology|Volume 23, Issue 7, P1889-1893, Jul. 1, 2012
2. WO2015063694
3. Yolanda M. Jacobo-Delgado et al; Peptides, Volume 142, 2021, 170580, https://doi.org/10.1016/j.peptides.2021.170580
4. Hatakeyama S et al, J Periodontal Res. 2010 April; 45(2):207-15. doi: 10.1111/j.1600-0765.2009.01219.x
5. WO2012/140504.
6. Punnapuzha S, Edemobi P K, Elmoheen A. Febrile Neutropenia. [Updated 2022 Feb. 10]. In: StatPearls [Internet]. Treasure Island (FL): StatPearls Publishing; 2022 January. Available from: https://www.ncbi.nlm.nih.gov/books/NBK541102/
7. Bodey G P, Buckley M, Sathe Y S, Freireich E J. Quantitative relationships between circulating leukocytes and infection in patients with acute leukemia. Ann Intern Med. 1966 February; 64(2):328-40. doi: 10.7326/0003-4819-64-2-328. PMID: 5216294
8. Meza L, Baselga J, Holmes F A, Liang B, Breddy J, for the Pegfilgrastim Study Group (2002) Incidence of febrile neutropenia (FN) is directly related to duration of sever neutropenia (DSN) after myelosuppressive chemotherapy. Proc Am Soc Clin Oncol 21: Abstract 2840
9. Lyman G H, Lyman C H, Agboola O, for the ANC Study Group (2005) Risk models for predicting chemotherapy-induced neutropenia. Oncologist 10: 427-437
10. Aapro M S, Cameron D A, Pettengell R, Bohlius J, Crawford J, Ellis M, Kearney N, Lyman G H, Tjan-Heijnen V C, Walewski J, Weber D C, Zielinskil C, European Organisation for Research and Treatment of Cancer (EORTC) Granulocyte Colony-Stimulating Factor (G-CSF) Guidelines Working Party (2006) EORTC guidelines for the use of granulocyte-colony stimulating factor to reduce the incidence of chemotherapy-induced febrile neutropenia in adult patients with lymphomas and solid tumours. Eur J Cancer 42: 2433-2453
11. Chelakkot, C., Ghim, J. & Ryu, S. H. Mechanisms regulating intestinal barrier integrity and its pathological implications. Exp Mol Med 50, 1-9 (2018). https://doi.org/10.1038s12276-018-0126-x
12. Taur Y, Pamer E G. The intestinal microbiota and susceptibility to infection in immunocompromised patients. Curr Opin Infect Dis. 2013 August; 26(4):332-7. doi: 10.1097/QCO.0b013e3283630dd3. PMID: 23806896; PMCID: PMC4485384.
13. JAMA. 2016 Feb. 23; 315(8): 801-810
14. Shock. 2016 July; 46(1): 52-59
15. Crit Care Med. 2011; 32:626-638)
16. Biochem Med (Zagreb) 2013; 23:107-111
17. Ridge J A, Glisson B S, Lango M N, et al. “Head and Neck Tumors” in Pazdur R, Wagman L D, Camphausen K A, Hoskins W J (Eds) Cancer Management: A Multidisciplinary Approach. 11 ed. 2008
18. Precision Medicine for Investigators, Practitioners and Providers, 2020
19. Batshaw et al. (2001) J. Pediatr. 138 (1 Suppl): S46-54; discussion S54-5
20. Roque et al. J Pharmacol Exp Ther. 2008 September; 326(3):949-56. Epub 2008 Jun. 23
21. US20080038374
22. WO/2008/073174
23. US2002-0076393
24. US2003-0109582
25. U.S. Pat. No. 7,311,925
26. Berge, et al., J. Pharm. Sci., 66, 1-19 (1977).
27. Protective Groups in Organic Synthesis (T. Green and P. Wuts, Wiley, 1999)
28. Pinto, M., M. D. Appay, P. Simon-Assman, G. Chevalier, N. Dracopoli, J. Fogh, and A. Zweibaum, 1982, Biol. Cell., 44:193-196
29. Nylén F, Miraglia E, Cederlund A, Ottosson H, Stromberg R, Gudmundsson G H, Agerberth B. 2013. Boosting innate immunity: Development and validation of a cell-based screening assay to identify LL-37 inducers. Innate Immun.

1. A compound of formula (I) for use in a method of treatment of a disease or condition in an animal, that can be treated by improvement or restoration of epithelial barrier function of the animal, wherein the compound is defined by the following formula: 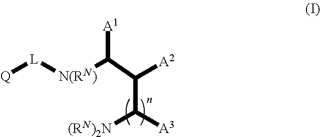
wherein:
Q is selected from Q1, Q2, Q3, Q4, Q5 and Q6: 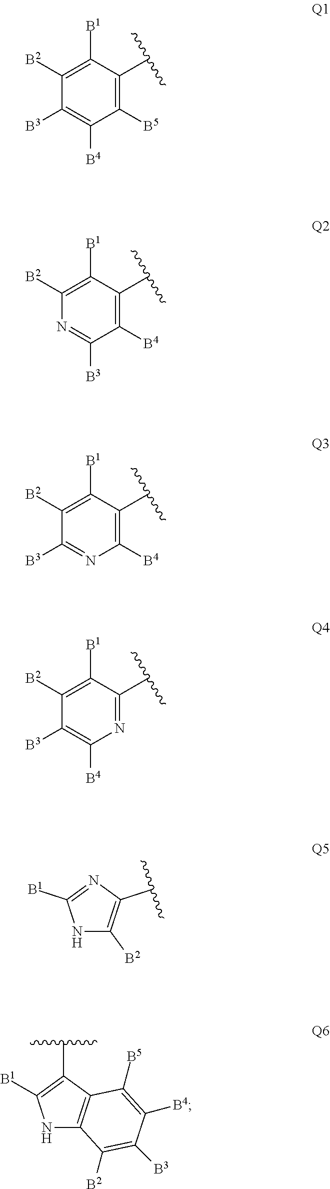
n is 0 or 1;
L is selected from —(CH₂)ₘ—, —C(═O)—, —(CH₂)ₘ—C(═O)—, —O—(CH₂)ₘ—C(═O)—, —O—C(═O)—(CH₂)ₘ—(C=O)—, —NH—C(═O)—, —NR—C(═O)—, —NH—(CH₂)ₘ—C(═O)—, —NR—(CH₂)ₘ—C(═O)—, —NH—C(═O)—(CH₂)ₘ—C(═O)—, —NR—C(═O)—(CH₂)ₘ—C(═O)—, —C(═O)—NH—(CH₂)ₘ—C(═O)—, and —(CH₂)ₘ—(CHR^{L})—C(═O)—, where m is an integer from 1 to 4;
A¹ and A², together with the atoms to which they are bound, form an optionally substituted C₆₋₁₄aryl group;
A³, if present, is selected from H and optionally substituted C₁₋₄alkyl;
R^{N} is selected from H and optionally substituted C₁₋₄alkyl;
one of B¹, B², B³, B⁴, and B⁵ is a group of formula —X—R^{X} and the others are independently selected from H and R^{B};
wherein each —R^{B} is independently selected from halogen, —CF₃, —R, —OH, —OR, —OCF₃, —C(═O)OH, —C(═O)OR, —C(═O)R, —OC(═O)R, —NH₂, —NHR, —NR₂, —NO₂, —C(═O)NH₂, —C(═O)NHR, C(═O)NR₂, —S(═O)R, —S(═O)₂R, —S(═O)₂NR₂, or —CN;
X is selected from a covalent bond or C₁₋₃alkylene;
R^{X} is selected from —H, R^{XX} or R^{XY};
wherein:
R^{XX} is halogen, —CF₃, —OH, —OR, —OCF₃, —C(═O)OH, —NO₂, —NH₂, —NHR, —NR₂, —C(═O)NH₂, —C(═O)NR₂, —S(═O)R, —S(═O)₂R, —S(═O)₂NR₂, or —CN; and
R^{XY} is a group of formula -L^{X}-R^{YY};
wherein L^{X} is selected from:
—NH—C(═O)—O—, —NH—C(═O)—NH—, —NH—C(═O)—, —NR—C(═O)—, —O—C(═O)—NH—, —O—C(═O)—O—, —O—(C=O)—, —C(═O)—NH—, —C(═O)—O—, and —C(═O)—;
and R^{YY} is selected from C₁₋₄alkyl, C₃₋₆cycloalkyl, —C₆₋₁₄aryl, -L^{Y}-C₆₋₁₄aryl, -L^{Y}-O—C₁₋₄aryl, —C₅₋₆heteroaryl, -L^{Y}-C₅₋₆heteroaryl, -L^{Y}-O—C₅₋₆heteroaryl, -L^{Y}-O-L^{Y}-C=N, -L^{Y}-O-L^{Y}-C=R, -L^{Y}-O-L^{Y}-C≡CH, -L^{Y}-O-L^{Y}-NR^{N}—C(═O)—R, -L^{Y}-O-L^{Y}-NH—C(═O)—R, -L^{Y}-O-L^{Y}-NR—C(═O)—R, -L^{Y}-C≡N, and -L^{Y}-C=R, -L^{Y}-C≡CH,
wherein -L^{Y}- is C₁₋₃alkylene and wherein each of said groups is optionally substituted;
R^{L} is selected from halogen, —R^{LL}, —CF₃, —OH, —OR^{LL}, —NO₂, —NH₂, —NHR^{LL}, —NR₂, —NH—C(═O)—R^{LL}, —NH—C(═O)—O—R^{LL} wherein R^{LL} is selected from —C₁₋₄alkyl, —C₃₋₆cycloalkyl, -Ph, -L^{L}-Ph, —C₅₋₆heteroaryl, -L^{L}-C₅₋₆heteroaryl wherein -L^{L}- is C₁₋₃alkylene.
and wherein each R is independently C₁₋₄alkyl.

2. The compound described in claim 1, for use in a method of treatment of a disease or condition in an animal that can be treated by preventing or reducing microbial translocation through the epithelial barrier of the animal.

3. The compound for use according to claim 1 or claim 2, wherein the epithelial barrier is the gastrointestinal epithelial barrier.

4. The compound for use according to claim 3, wherein the intestinal epithelial barrier is the intestinal epithelial barrier.

5. The compound described in any one of the above claims, for use in a method of treatment and/or prevention of a microbial infection in an animal.

6. The compound described in any one of the above claims, for use in a method of treatment and/or prevention of febrile neutropenia in an animal.

7. The compound for use of any one of the above claims, wherein the animal has been or is being treated for cancer.

8. The compound for use of any one of the above claims, wherein the animal is the recipient of an organ transplant, optionally wherein the animal is immunocompromised.

9. The compound for use of any one of the above claims, wherein the animal has a low neutrophil count.

10. The compound described in any one of the above claims, for use in a method of treatment and/or prevention of sepsis in an animal.

11. The compound described in any one of the above claims, for use in a method of treatment and/or prevention of bacteremia or Fungemia in an animal.

12. The compound described in any one of the above claims, for use in a method of treatment and/or prevention of mucositis in a subject.

13. The compound described in any one of the above claims, for use in a method of treatment and/or prevention of microbiome dysbiosis in an animal.

14. The compound described in any one of the above claims, for use in a method of treatment and/or prevention of a condition selected from: Atopic Dermatitis, Asthma, Allergic Rhinitis, Chronic rhinosinusitis, Eosinophilic Esophagitis, Meningitis, COPD, Periodontitis Bronchitis, Eczema, Inflammatory Bowel Disease, Coeliac Disease, Leaky Gut Syndrome, Alzheimer Disease, Parkinson Disease, Chronic Depression, Autism, Diabetes, Obesity, Non-Alcoholic Steatohepatitis, Autoimmune Hepatitis, Liver Cirrhosis, Rheumatoid Arthritis, Multiple Sclerosis, Systemic Lupus Erythematosus, Ankylosing Spondylitis, Intestinal Tissue Inflammation, Non alcoholic fatty liver disease and Necrotizing Enterocolitis.

15. The compound described in any one of the above claims, for use in a method of treatment and/or prevention of diarrhoea, for example traveller's diarrhoea.

16. The compound for use according to any one of the above claims, wherein administration of the compound improves or restores epithelial barrier function in the animal, and/or prevents or reduces microbial translocation through the epithelial barrier of the animal.

17. The compound for use according to claim 16, wherein the compound is capable of preventing microbial translocation from the gut of the animal to the kidney, liver, spleen or other organs and vascular beds via the circulatory system.

18. The compound for use according to claim 16, wherein the compound is capable of improving, restoring or maintaining tight junction function of the epithelial barrier of the animal.

19. The compound for use in a method of treatment and/or prevention of a microbial infection according to any one of the above claims, wherein the microbial infection is selected from the group consisting of bacterial, viral, prion, protozoal and fungal infections.

20. The compound for use according to claim 19, wherein said microbial infection is caused by a microbial species of a genus selected from the list consisting of: Yersenia, Salmonella, Shigella, Campylobacter, Clostridium; Heliobacter; Mycobacterium, Pseudomonas, Haemophilus, Moraxella, Escherichia, Neisseria, Streptococcus, Staphyllococcus, and Norovirus.

21. The compound for use according to claim 19, wherein said microbial infection is caused by Klebsiella pneumonia, Escherichia coli, Enterobacter spp., Serratia spp., Proteus spp., Providencia spp., Morganella spp., Enterococcus faecium, Staphylococcus aureus, Helicobacter pylori, Acinetobacter baumannii, Pseudomonas aeruginosa, Campylobacter (e.g. Campylobacter jejuni), Salmonella spp., Neisseria gonorrhoeae, Streptococcus pneumoniae, Haemophilus influenzae, Shigella spp.

22. The compound for use according to claim 19, wherein said microbial infection is caused by Nairovirus, Marburg Virus, Ebola virus, Coronaviridae, Mammarenavirus, Henipavirus, Phlebovirus, Chikungunya, Alphavirus (Togavirus), Zika, and Dengue or other Flavivirus.

23. The compound for use according to claim 19, wherein said microbial infection is caused by Yersenia enterocolitica, E. coli, Clostridium difficile, Helicobacter pylori, Mycobacterium tuberculosis, Haemophilus influenza, Moraxella catarrhalis, Pseudomonas aeruginosa, Staphyllococcus aureus, Group A and B Streptococcus, HIV, RSV, influenza virus, Herpes or Hepatitis viruses.

24. The compound for use according to claim 19, wherein said microbial infection is caused by a bacterial strain resistant to direct-acting antibiotic treatment.

25. The compound for use according to any one of the above claims, wherein the compound is a compound according to formula (Ia):

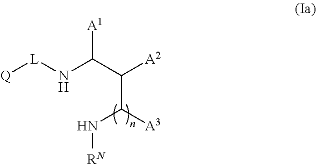

26. The compound for use according to any one of the above claims, wherein A¹ and A², together with the atoms to which they are bound, form an optionally substituted phenyl, naphthalene or heteroaryl group.

27. The compound for use according to any one of the above claims, wherein A¹ and A², together with the atoms to which they are bound, form a phenyl group.

28. The compound for use according to any one of the above claims, wherein R^{N} is H.

29. The compound for use according to any one of the above claims, wherein n is 0.

30. The compound for use according to any one of the above claims, wherein Q is Q1.

31. The compound for use according to any one of the above claims, wherein B³ is —X—R^{X}.

32. The compound for use according to claim 30 or claim 31, wherein B¹, B², B⁴ and B⁵ are all H.

33. The compound for use according to any one of the above claims, wherein L is selected from —(CH₂)ₘ—, —C(═O)—, —NH—C(═O)—, and —NR—C(═O)—.

34. The compound for use according to claim 32, wherein L is —C(═O)—.

35. The compound for use according to any one of the above claims, wherein X is a covalent bond.

36. The compound for use according to any one of claims 1-34, wherein X is C₁₋₃alkylene.

37. The compound for use according to claim 36, wherein X is —CH₂—.

38. The compound for use according to any one of the above claims, wherein R^{X} is —R^{XY}.

39. The compound for use according to any one of the above claims, wherein Lx is independently: —NH—C(═O)—O—, —NH—C(═O)—, —NR—C(═O)—, or —O—(C=O)—.

40. The compound for use according to claim 38, wherein L^{X} is independently —NH—C(═O)— or —NH—C(═O)—O—.

41. The compound for use according to any one of the above claims, wherein R^{YY} is independently: -L^{Y}-O-L^{Y}-C═H, -L^{Y}-O-L^{Y}-NH—C(═O)—R or -L^{Y}-C≡CH.

42. The compound for use according to any one of claims 1-40, wherein R^{YY} is -L^{Y}-C₅₋₆heteroaryl.

43. The compound for use according to any one of the above claims, wherein -L^{Y}- is —CH₂—.

44. The compound for use according to any one of claims 1 to 40, wherein R^{YY} is substituted with one or more substituents selected from: —F, —Cl, —Br, —I, —R, —CF₃, —OH, —OR, —OCF₃, —NO₂, -L^{YY}-OH, -L^{YY}-OR, —NH₂, —NHR, —NR₂, -L^{YY}-NH₂, -L^{YY}-NHR, -L^{YY}-NR₂, —CO₂H, —CO₂R, -L^{YY}-CO₂H, -L^{YY}-CO₂R, -Ph, and -L^{YY}-Ph-, wherein L^{YY} is C₁₋₃alkylene.

45. The compound for use according to any one of the above claims, wherein the compound is selected from: 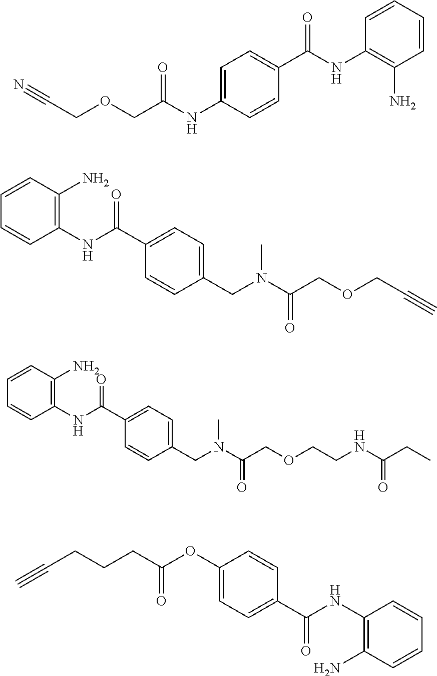
and Pyridin-3-ylmethyl (4-((2-aminophenyl)-carbamoyl)benzyl)carbamate (Entinostat):

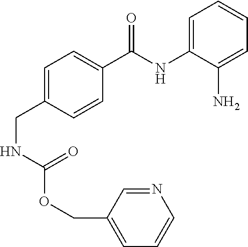 [SMILES: Nc1ccccc1NC(=O)c1ccc(CNC(=O)OCc2cccnc2)cc1]

46. The compound for use according to any one of the above claims, wherein the method comprises administration of the compound to the animal in an effective amount of the compound, wherein the weekly or daily dosage is between 10 μg to about 1 g which is optionally split into doses given 1, 2 or 3 times.

47. The compound for use according to claim 46 wherein the dosage is between 0.025 mg and 500 mg.

48. The compound for use according to claim 47, wherein the dosage is between 0.1 mg and 250 mg.

49. The compound for use according to any one of claims 46-48, wherein the compound is administered at a dose of at least 5 mg/kg, at least 10 mg/kg, at least 15 mg/kg, at least 20 mg/kg, at least 25 mg/kg, at least 30 mg/kg, at least 35 mg/kg, at least 40 mg/kg, at least 45 mg/kg, at least 50 mg/kg, at least 55 mg/kg, at least 60 mg/kg, at least 65 mg/kg, at least 70 mg/kg, at least 75 mg/kg, at least 80 mg/kg, at least 85 mg/kg, at least 90 mg/kg, at least 95 mg/kg, or at least 100 mg/kg.

50. The compound for use according to claim 49, wherein the compound is administered at a dose of at least 50 mg/kg.

51. The compound for use according to any one of claims 46-50, wherein the compound is administered at a dose of less than 150 mg/kg.

52. The compound for use according to any one of the above claims, wherein the treatment is a combination treatment, wherein the compound is used in combination with any one or more of: an antibiotic; isoleucine or active isomers or analogs thereof; a vitamin D type compound.

53. The compound for use according to any one of the above claims, wherein the compound is present in a pharmaceutical composition comprising as an active ingredient said compound, in addition to at least one pharmaceutically acceptable excipient.

54. The compound for use according to claim 53, wherein the pharmaceutical composition is formulated as an oral dosage form.

55. The compound for use according to claim 54, wherein the oral dosage form is selected from a tablet, a capsule, a solution, a suspension, a powder, a paste, an elixir, a syrup, and a lozenge.

56. The compound for use according to claim 54 or claim 55, wherein the oral dosage form comprises in the range of about 0.01-1000 mg of said active ingredient.

57. The compound for use according to claim 53, wherein the pharmaceutical composition is formulated as an inhalation dosage form.

58. The compound for use according to any one of the above claims, wherein the method comprises:
(1) administration to the animal of an antibiotic for 1 or 2 days with or without the compound; followed by
(2) administration to the animal of an effective amount of the compound for a further 2, 3, 4, 5 or more days.

59. A method of treatment as described in any one of the above claims.

60. Use of a compound described in any one of the above claims in the preparation of a medicament for use in a method of treatment as described in any one of the above claims.

61. A compound, use or method as claimed in any one of the preceding claims wherein the animal is a mammal, preferably wherein the animal is a human.

62. A compound, use or method as claimed in any one of the preceding claims wherein the animal is selected from fish, dogs, cats, cows, horses, deer and poultry including hen, turkey, ducks, geese, and household pets such as birds and rodents.

63. A compound selected from: [chemical structure]
